(19) United States
(12) Patent Application Publication
(10) Pub. No.: US 2025/0285853 A1
(43) Pub. Date: Sep. 11, 2025

(54) MULTI-PRESSURE CHEMICAL IONIZATION (MPCI) SYSTEM, MASS SPECTROMETER AND METHOD USING THE SAME

(71) Applicant: KARSA OY, Helsinki (FI)

(72) Inventors: Aleksei SHCHERBININ, Helsinki (FI); Jyri MIKKILÄ, Helsinki (FI); Jussi KONTRO, Helsinki (FI); HJ JOST, Helsinki (FI); Henning FINKENZELLER, Helsinki (FI)

(21) Appl. No.: 18/658,763

(22) Filed: May 8, 2024

Related U.S. Application Data
(60) Provisional application No. 63/563,255, filed on Mar. 8, 2024.

(51) Int. Cl. H01J 49/00; G01N 33/00

(52) U.S. Cl. CPC H01J 49/0095; G01N 33/0027

(57) ABSTRACT

The present disclosure of the invention concerns embodiments directed to a multi-pressure chemical ionization multi-ion identification device (MPCI MION device), a system and method using the same to utilize chemical ionization (CI) in multiple adduct formation from the substances in the sampled gas of a gas sample being addressed to be analyzed in a mass analyzer. The multi-pressure multi-ion identification (MPCI MION) device comprises a buffering region to have the sample flow turbulence decayed before the sample flow entrance to the low pressure ionization regions (IR(A)), IR(B), (IR(B′), (IR(C′), IR(D′)) (IR(E′)) utilizing chemical ionization by reagents from an ensemble of reagent ion towers (R1, R2, R3, R4, R5, R6, R7, R8, R9, R10, R11, R12, R13, R14, R15, R16, R17, R18,).

### TECHNICAL FIELD OF THE INVENTION

Generally, the present invention relates to ion identification from a medium being sampled to the ion identification device. In particular, the present disclosure of embodiments of the invention pertains improvements in overall performance to a multi-ion identification device to determine a composition of gaseous species in the sampled medium to be analyzed to identify the ions and so to determine the composition of the sample. The present invention relates also to such a program product that is used in the control of such an improved system for the ion identification by the multi-pressure ion identification device. The present invention relates also to a system to produce a database being formed from the measurement results being made by the system of the present disclosure.

### BACKGROUND

Determination of various gaseous compositions comprising organic constituents in the gases has been made in the history in 150 years as such. One example of an early composition study relates to Breath analysis dates to 1874 when Francis E. Anstie made the first observations that alcohol could be detected in excreted breath air. This was of little functional use at the time, but the first breathalyzer was invented in 1927 and the first practical roadside test was commercialized in 1931 with the first electronic version being developed in 1967.

In current clinical settings some diagnoses can be aided through breath screening devices (e.g. asthma), however, the promise of accurate diagnosis of many conditions, just by blowing into a tube, has been much vaunted, but never achieved, leading to certain skepticism within clinical community to have such a device to assist to provide sufficient compositional information to be used in the analysis by the professional persons.

Instant diagnosis and screening of medical conditions has always been a dream of science fiction, from the tricorder in ‘Star Trek’ to the diagnostic bed, a staple of many sci-fi stories. These tools, even today, still seem to be decades away, but recently breath analysis has been showing a promise to have a diagnose being based on data obtained by certain limited conditions in a non-invasive way. There is a question what prevents devices to breath analysis from becoming these truly universal diagnostic tools for the professional persons? For one, chemical complexity of human breath as exhaled is beyond the limitation of current analysis techniques, such as Gas Chromatography (GC) and Proton Transfer Reaction Mass Spectrometry (PTR-MS). Although these devices and systems have excellent analytical capabilities and have been used for decades they seem to struggle when it comes to detecting more functionalized and less volatile compounds in gas sample, a class of molecules that may in fact become the cornerstones of breath analysis data to be used. However, as these tools can only detect a certain range of compounds, and only within a limited scope, this is one of the limiting factors that has slowed the development of breath analysis as a usable and universal tool for professional persons to make diagnostics. More information on the limitations of these systems come mainly from (i) sampling of semi-to-low-volatile material and (ii) limit of detection due to poor ionization efficiency, for example.

Artificial intelligence was also once a science fiction trope that showed how the halcyon days of the future would be cushioned by AI support, anticipating the needs of users and providing information and support on a just in time basis. Whereas this AI was unthinkable 20 years ago, nowadays AI and machine learning, that can teach itself and become more useful with little human input, is becoming standard. With the many examples of AI that can be seen in modern times, such as better traffic management, to entertainment services, to enhanced fraud detection, these all need one common element to make them work. Data. Lots of data.

A decade ago, breath analysis was a promising emerging technology that aspired to become the new screening tool in the medical arsenal to detect diseases that are hard to diagnose or otherwise screen for. Some progress has been made over the past years, but the promise remains unfulfilled and medical practitioners and clinicians have become sceptical¹. A functional breath test is a convenient and minimally invasive method for detection of certain compounds in exhaled breath which could be used for material for producing data for diagnosis of certain conditions and health risks. Currently, only a few breath analysis tools have made it to mainstream clinical practice.

Recent systematic review and meta-analyses of Volatile Organic Compounds (VOC) based breath tests for cancer diagnosis revealed lack of methodological standardization while mentioning that tests have potential for non-invasive cancer diagnosis². A framework for conducting and reporting future studies investigating the role of VOCs in cancer diagnosis was proposed by the authors of the review. However, research have shown that previous attempts may have had methodological problems that could lead to overestimation of VOC-based breath tests performance³. This criticism focused mainly on methodological problems. The analysis of VOCs that differ between two groups of study participants (e.g. diseased and healthy) is often the primary aim of such studies, while the origin and the pathophysiological significance of the identified marker compounds have been frequently neglected⁴. Breath-atlas proposes radically different approach where the first question asked is: what chemical compounds constitute normal human breath?

Human breath contains hundreds of different volatile compounds and the distribution varies from one person to another. Some compounds in exhaled breath are the results of environmental exposure, others are generated by the normal metabolism of the body, and still others are the products of bacterial or viral infections or other medical conditions.

Exhaled breath is a complex gas mixture with hundreds (potentially many thousands) of analytes and the concentrations of the biomarkers are often very low (in the part-per-billion (PPB) or part-per-trillion (PPT) range, from those that are known). To target virtually every gas-phase chemical compound, a collection of ionization reagents is needed. With increasing functionalization of a molecule, simple molecular parameters become less well defined. One simple example is offered by amino acids, which have characteristically both acidic and basic functionalities, with the acid-base behavior changing depending on the structure of the rest of the molecule.

There is widespread need for an advanced online chemical analysis of gaseous samples: from detecting explosives, to screening food samples for presence of pesticides, to environmental monitoring, to diagnosing diseases from breath. There is one major shortcoming that hinders chemical technology to achieve that and become more than a tool for research. Real world problems usually include detecting multiple compounds from very different chemical classes, for instance from very polar and acidic to less functional compounds. Each technique like PTR, GC or APCI has it's limitations in terms of what kind of molecules they are best to distinguish and identify and changing between the set-ups is hard and laborious, and may take hours or days. Present invention proposes a way to overcome this problem and provide set of tools for unbiased and standardized analysis of gaseous samples for chemical composition.

Although a wide range of detectable compounds at high sensitivity and possibility to analyze composition of analyte by multiple ions can be provided, simple non-polar compounds due to their non-polar nature and low proton affinities are typically lost from the results because of high-collision rate at atmospheric pressure and consequent loss of the charge.

There are known solutions introduced as such, to detect these compounds (for example PAHs, i.e. polycyclic aromatic hydrocarbons and BTEX, i.e. Benzene, Toluene, Ethylbenzene and Xylene or alike and their derivatives) a multitude of low-pressure (operating in pressures 1-10 mbar) PTR (Proton Transfer Reaction) ionization systems as such.

### SUMMARY OF THE INVENTION

The objective is to at least alleviate the problems described hereinabove not satisfactorily solved by the known arrangements, and to provide a feasible multi-ion identification device to be used in gaseous composition sample analysis, to reveal also such more functionalized and less volatile compounds to be identified and analyzed for the data about the composition and the abundances of those molecules in it, as well as to provide information on more volatile less functionalized compounds simultaneously without the need to change the setup and interrupt sampling. Accordingly embodiments of the present disclosure build upon KARSA's previous U.S. Pat. No. 11,710,625, which is incorporated here by reference in its whole entirety, the specification, claims and drawings as such, the improvements for the improved overall performance of the system of the present disclosure also overcomes the problem of requiring multiple mass spectrometers to analyze wide range of relevant compounds from non-functionalized group such as PAH (polycyclic aromatic hydrocarbons) to polar compounds, basic or acidic, including in the operating range of the embodied system, and method of using of the system as combines high-pressure and low-pressure ionization schemes and so to overcome prior limitations by opening a more universal and efficient use of powerful mass spectrometry devices based systems according to the present disclosure of embodiments.

Mass spectrometers are analytical instruments that can be used to analyze the chemical composition of air with high precision. The problem previously has been that they only measure ions, which means that the molecules need to be charged (ionized) first. Several ionization techniques have been developed, each one with their own characteristics and applications.

Chemical ionization (CI) is a method in which a reagent chemical is used to produce reagent ions, that are mixed with the air sample. It is a soft ionization method using such mechanism, meaning that the molecules of interest will stay intact and cluster with the reagent ion. This greatly helps with detection, because the elemental composition of the target molecule generally does not change.

Another important aspect of chemical ionization is selectivity. Certain reagent ions tend to cluster only with molecules having distinct properties. Selectivity is both a good and a bad thing. It allows for great sensitivity when the target molecule has the tendency to cluster with the selected reagent ion, because majority of the molecules in the air are not ionized and thus noise level is low.

However, the obvious drawback is that a certain reagent only works for limited number of molecules, and as such, it is not a suitable method for detecting a broad range of chemicals with varying properties.

Another complication of using conventional CI and SESI (Secondary Electrospray Ionization) is neutral reagent vapors/droplets mixing with sample flow, which alters the sample chemistry and complicates using more than one ionization scheme.

The aforesaid objective is achieved by the embodiments of a multi-pressure multi-ion identification device according to the present disclosure, to be used in a system and method of comprehensive data analysis in accordance with the present disclosure of the invention. It is in the interest of the applicant to build a ground-breaking tool for online gas phase analysis that will not only improve detection of the known compounds in air, such as found in exhaled breath, but open a possibility to study molecules that has been only anticipated in theory and never before measured in practice so far in a systemic and standardized method and provide researchers using said devices with a knowledgebase on gas phase chemistry that will evolve with time, so that also the wide range of relevant compounds from non-functionalized group such a PAH (polycyclic aromatic hydrocarbons) to polar compounds, basic or acidic can be analyzed without a significant loss of such molecules.

It is the machine learning algorithms (such as deep learning) to be used in analysis of this data and the comparison of data in huge sets that allows the software to recognize patterns of the constituents and suggest to react accordingly, in order to deliver analysis of complex patterns within sample chemistry and create classification predictions, such as deriving underlying conditions that lead to particular chemical composition.

The disruptive approach can be made by employing an embodied online system of the present disclosure to provide Multi-Pressure Chemical Ionization Multi Ion Mass Spectrometer i.e. MPCI MION Multi Ion Orbitrap Mass Spectrometer (i.e. MPCI-MIONOT-MS) or any other suitable ion detection device as a detector for the mass analysis as a device with mass spectrometer functionality, such as IMS which simultaneously utilizes multiple selective ion chemistries both in negative and positive modes of detection, which enables the MPCI-MION-system to detect a wide range of very different chemical classes with unprecedented sensitivity and selectivity. Thus, providing a possibility of unbiased and comprehensive chemical analysis of gaseous samples.

According to an embodiment, a Multi-Pressure Chemical Ionization Multi Ion Orbitrap Mass Spectrometer (MPCI MION-OT-MS) is capable of detecting virtually every gas phase molecule potentially present in human breath at unprecedented sensitivity. Using an embodied system comprising a MPCI MION OT-MS, will develop a gas sampling methodology suitable for large scale quantitative analysis of the composition breath as an example (accounting for exhaled volume, relative humidity and other underlying conditions affecting the spectral signal). But also suitable for example for ambient environmental measurements.

At the same time, other tools can be used for collecting comprehensive auxiliary metadata to a database being developed, allowing for multifactor analysis of the acquired breath spectra and their composition. Next, further measurement data is collected. This innovative and unique approach according to the embodiments of the present disclosure gives the possibility for detection of a wide range of chemical compounds previously not detected with conventional tools. This unbiased sampling of the compositional information in the sample, such as breath for example, reveals the components for optimization the resolution and predictivity in a similar way as broad expression genomic and expression-based chemistry for example cancer analysis was needed to enable new, would provide means for personalized treatments being order as based on the analysis made by the medical professionals aided by the data being created by embodiments of the present disclosure.

Specific algorithms can be used in the embodied system with a dedicated configuration to apply ‘big data’ techniques and Machine Learning Algorithms (MLA) to analyze the data obtained from the samples being sampled by the embodiments of the present disclosure, i.e. that has been collected through the breath sampling and the auxiliary complementary data and attempt to correlate specific chemicals found in breath with different sociomedical attributes related to the composition. Embodiments can make such work done applying multivariate statistical methods for identification of patterns of breath markers in gas compositions of both healthy individuals and for the study of the gas compositions of breath-based disease networks (e.g. asthma, cancer, and other lung pathologies) to provide the data for professionals to make diagnosis as based on the data.

The aforesaid objective(s) are achieved according to the present disclosure of the embodiments of the invention as claimed in claim 1.

An MPCI MION multi-pressure multi-ion identification device according to an embodiment of the disclosure of the present invention in combination with one or more embodiments comprises in the Multi pressure chemical ionization multi-ion identification device, (MPCI-MION device), a sample introduction port for sample with analyte molecules therein, said sample introduction port being followed by a stack of ionization stages, wherein said stack comprises at least one ionization stage in atmospheric and/or higher pressure followed by a number of underpressure ionization stages in series of reducing operation pressure towards an exit to a mass analysis device such as a mass spectrometer for a mass spectrum acquiring.

According to an embodiment a pressure ratios (i.e. such as for example P(A)/P(B), P(B)/P(B′), P(B′)/P(C′), P(C′)/P(D′) P(D′)/P(E′)), between a first stage in a high pressure and/or in atmospheric pressure conditions (i.e. P(A) or P(B)) and a next following stage in series towards the mass analysis device, i.e. a stage in a lower pressure can be selected to at least one of the following decimal fractions from: 1, 10⁻¹, 10⁻², 10⁻³, 10⁻⁴, 10⁻⁵, 10⁻⁶, 10⁻⁷, 10⁻⁸, 10⁻⁹, i.e. such ratios as P(A) or P(B) to P(B′), etc.

Accordingly, the pressure ratio of two successive next following stages), P(B)/P(B′), P(B′)/P(C′), P(C′)/P(D′) P(D′)/P(E′), in a lower pressure can be selected for the pressure ratio as the following decimal fractions from 1, 10⁻¹, 10⁻², 10⁻³, 10⁻⁴, 10⁻⁵, 10⁻⁶, 10⁻⁷, 10⁻⁸, 10⁻⁹, so that the last stage before the entry to the mass analysis device matches to the pressure in the conditions in the mass analysis device. As an example of the ratios being illustrated as P(A)/P(B), P(B)/P(B′), P(B′)/P(C′), P(C′)/P(D′) P(D′)/P(E′), the conditions discussed later in the application text parts, According to an embodiment variant the pressure ratio of the first high pressure (or as atmospheric stage) as well as the next following stages can be selected in an intermediate range of the above indicated any two successive decimal fractions, i.e. for example such as 0.2 or 0.03 or 0.00007, as no intention to limit any of the ratios such as P(A)/P(B), P(B)/P(B′), P(B′)/P(C′), P(C′)/P(D′) P(D′)/P(E′), only to the shown intermediate decimal ratios.

However, according to an embodiment an alternative variant the high pressure is a pressure that is considered under 10 bar, in an embodiment variant 1 bar, or in a further variant under 500 mBar, but anyhow above 200 mBar.

According to an embodiment in combination with one or more embodiments of the present disclosure, each ionization stages comprising a ionization-stage-specific number of ion injection towers, each said ion injection tower being selected for a dedicated reagent emission in the ionized form at the entry to the corresponding ionization stage, for chemical ionization of analytes from the sample, in such a ionization stage in said ionization stage specific pressure and temperature condition.

According to an embodiment of the present disclosure, the MPCI-MION device in combination to one or more embodiments of the present disclosure comprises in each ionization stage with its first plurality of reagent ion towers (R1, R2, R3) to provide dedicated ionization reagent ions each, said each ionization stages are positioned into a stack as stacked to a planar geometry with planes (IR(A), IR(B), IR(B′), IR(C′), IR(D′), IR(E′),) each ionization stages as mutually parallel while perpendicular to sample flow entrance direction to ionization region of the Multi pressure chemical ionization multi-ion identification device (100).

According to an embodiment of the present disclosure, the Multi pressure chemical ionization device (MPCI-MION device) according to one or more embodiments of the present disclosure comprises in each ionization stage has a predetermined thickness in the sample flow direction correspond the timescale of chemical ionization in the pressure and temperature conditions of the ionization stage to provide a stage specific reaction time for the ions emitted from the respective ion towers to chemically ionize analyte molecules in the sample, the so formed adducts being transported from the ionization region to next stage or to a mass spectrometer port for mass analysis of the species of the adducts.

According to an embodiment of the present disclosure, the MPCI-MION device according to one or more embodiments of the present disclosure, has been configured so that each ionization reagent of the respective reagent ion injection tower in same common plane is configured to operate according to same ion production mechanism to provide respective reagent ions from a dedicated reagent ion tower.

According to an embodiment of the present disclosure, the MPCI-MION multi-ion identification device according to one or more embodiments of the present disclosure is configured to ionize reagent molecules to reagent ions in the ion towers by the ionization mechanism of a reagent ion tower that is at least one of the following: X-ray, soft-X-ray, corona discharge, electrospray, xenon, vacuum UV-lamp, based ionization mechanism.

According to an embodiment variant, also direct ionization processes, such as direct photoionization, corona discharge and Townsend ionization to the lower pressure ionization condition can be used, in parallel to the chemical ionization, in the low pressure units/stages, in applicable part.

According to an embodiment of the present disclosure, at least one of the sheath flows is made to occur in laminar flow regime. According to an embodiment variant, the sheath flows are controlled to the laminar flow profile shape by equalizing their velocities. According to an embodiment, in an embodiment of the present disclosure, there is a flow shaper (i.e. a mesh or a number of consecutive meshes in the flow direction) for making each sheath flow laminar, to further fine tune the shape of the sheath flows to make sure that each sheath flow has laminar character in the laminar flow regime.

A skilled person in the art knows that the sample flow laminarization at the entry to the ionization regimes can be in the atmospheric pressure conditions with the respective stages. In consecutive low pressure stages the laminarization can be performed in suitable part, provided that the influence to the adduct loss would be predictably stable, when the sample is entering from a compartment to another in the respectively reducing pressure conditions.

According to an embodiment, a humid gas sample can be dried by a dryer as means for drying the sample flow by diffusional drying.

According to an embodiment of the present disclosure, in an embodied MPCI-MION multi-ion identification device according to one or more embodiments of the present disclosure, the polarity of a reagent ion tower produced ions are adjustable to positive or negative ions. According to an embodiment variant, a reagent ion tower can be controlled to neutral polarity, i.e. non-positive or non-negative polarity in addition to turn off and on when applicable.

The polarity can be changed by switching the charging mechanism for example, but also in addition by filtering the non-wanted ions by electric field out of reach of the exit of the reagent ion tower in question.

According to an embodiment of the present disclosure, in an embodied MPCI-MION multi-ion identification device according to one or more embodiments, at least one of the reagent ion towers comprises a filter to filtrate multiply charged reagent agents or unwanted polarity carrying reagent ions away from propagation for entry to the ionization region in the apparent stage. According to an embodiment, the filter is based on electrostatic fields. According to an optional embodiment, the filter is based on ion trap with alternating electric field.

According to an embodiment, the ion trap filter has in addition to alternating electric field an electrostatic field arranged to trap reagent ions to the ion trap.

According to an embodiment of the present disclosure, in an embodied MPCI MION multi-ion identification device according to one or more embodiments the ionization region comprises a round cylindrical symmetry with a centerline as a symmetry center of the stack. According to an embodiment, the multi-ion identification device comprises a buffering region. According to an embodiment, the buffering region has same symmetry axis as the ionization region. According to an embodiment, the buffering region has a length sized to flow velocity for a sample flow turbulence decay to laminar flow regime for the sample flow at the entry to ionization region. According to an embodiment, each sheath flow velocity at the buffering region entrance to enter ionization region has been matched to settle to laminar flow conditions. According to an embodiment each sheath air flows are adjusted to the flow velocity of the sample flow velocity. According to an embodiment, the adjustment is made by the control unit, according to an embodiment by the user, but according to an optional embodiment by the software arranged to adjust sheath flow valve as a sheath flow actuator.

According to one or more embodiments of the present disclosure, the underpressure compartments i.e. low-pressure stages/units, the length is selected to make sufficient the wanted ionization extent for the prevailing pressure conditions and the ionization time scale to occur in such.

According to an embodiment of the present disclosure, in an embodied multi-ion identification device according to one or more embodiments, the reagent ion towers in the same stage are aligned into a corresponding plane, and have an off-set (α) to deflect from the direction of the ionization region centerline along the respective emitting lines pointing radial directions.

An MPCI MION multi-ion identification system according to an embodiment of the present disclosure comprises:
at least one MPCI-MION multi-ion identification device according to one or more embodiments of the present disclosure of the invention,
at least one control unit to control the MPCI-MION multi-ion identification system and its actuators for the operation in mass analysis of adducts formed from the constituents of the sample,
a mass spectrometer to make said mass analysis,
a database to store mass analysis results.

Embodied system according to the present disclosure is also referred as MPCI MION-system.

According to an embodiment of the present disclosure of the invention, an embodied multi-ion identification system according to one or more embodiments of the present disclosure comprises a software packet configured to control operation of the MPCI MION multi-ion identification system. According to an embodiment, the software packet comprises the software of one or more control units, to control the actuators of the embodied system and/or its parts. According to an embodiment, the control unit is arranged to maintain the flows of the MPCI MION multi-ion identification device, voltages and/or the environmental quantities, such as pressures and temperatures in the stages of the stack, as well as to control the radiation source in the ionization in the ion towers, etc. According to one or more embodiments of the present disclosure, the control unit is arranged to operate the spectrometer as a system element. According to an embodiment variant, the software packet is additionally configured to constitute a database for mass spectra measurement results, obtained by the mass spectrometer in control of the control unit in measurements. According to an embodiment, the software packet is configured to provide a machine learning algorithm and number of statistical tools, which can have a self-learning packet for data analysis to make cluster analysis and/or to find marker substances from the composition data of the samples and/or mass spectrometer signals.

According to an embodiment of the present disclosure of the invention, an embodied MPCI-MION multi multi-ion identification device uses such a MPCI-MION system that comprises such a software packet (SW) that is configured to make group analysis from the clusters to find and deduce marker substances and/or newly found molecules, such as HOMs for example, from the results. These HOMs (Highly Oxidized Multifunctional organic molecules) can comprise hundreds of different types of molecules that were seldom detected, if never before at all, as components of secondary aerosol.

According to an embodiment of the present disclosure of the invention, an embodied software packet for the embodied multi-ion identification system comprises at least one of the following: a machine learning algorithm, a neuron network solver for optimization of cluster analysis, an artificial intelligence algorithm, such as penalized linear LARS, an elastic net regressions algorithm, random forests and recursive feature elimination algorithm.

An embodied software packet according to an embodiment of the present disclosure comprises configuration to operate and control the MPCI.MION system and its elements and devices according to an embodied method in combination to one or more embodiments of the present disclosure.

According to an embodiment, the system comprises an ion detector to detect ions, said ion detector as embodied for example as Ion Mobility Spectrometer, (IMS), or as mass spectrometer, such as a MPCI-Multi Ion Multi Pressure Chemical Ionization Orbitrap Mass Spectrometer (MPCI MION-OT-MS) being configured to simultaneously utilize multiple selective ion chemistries both in negative and positive modes of detection.

According to an embodiment of the present disclosure of the invention, an embodied MPCI MION multi-ion identification system comprises such a mass analysis device, for example as a mass spectrometer as a system element, that is a Multi Ion Multi Pressure Chemical Ionization Orbitrap Mass Spectrometer (MION MPCI-OT-MS) being configured to simultaneously utilize multiple selective ion chemistries both in negative and positive modes of detection.

According to an embodiment of the present disclosure of the invention, an embodied MPCI MION multi-ion identification system comprises in the system configuration to detect extremely low vapor pressure, highly oxidized multifunctional organic molecules (HOM) from the sample.

A method of identifying substances from a gas sample by using a MPCI MION-system according to an embodiment comprises:
using an MPCI MION multi-ion system (MPCI-MION system) according to one or more embodiments of the present disclosure, the method comprising:
   sampling a gas sample into a sample flow of the MPCI multi-ion identification device of the MPCI MION system,
   allowing turbulence to decay to laminar flow conditions of the sample flow in a buffering region of the MPCI-MION multi-ion identification device in the first stage,
   protecting the gas sample by at least one or two sheath flows at least in the buffering region,
   charging the gas sample constituents by reagent ion molecules formed for use in chemical ionization of said gas sample constituents to form adducts in corresponding stages with stage specific pressure and temperature,
   alternatively, charging happens with charge transfer (electron capture, proton transfer etc.)
   allowing the adduct to form from the gas sample constituent analytes and reagent ion molecules in corresponding stages in the pressure conditions therein,
   leading the adducts to mass spectrometer for mass analysis,
   identifying the adducts and the gas sample constituents,
   storing to a database the identified gas sample results identified constituents.

According to an embodiment, in combination with one or more embodiments, comprises using MPCI MION device as a system element of MPCI MION system in several ionization modes to provide chemical ionization to the analyte molecules in respective pressure conditions by one or more ion towers present in the stages in question, wherein the method comprises selecting of the ionization adduct forming Mode X, in conduct of a control unit controlling the method procedure, wherein the Mode comprises at least one or modes of the following Model, Mode 2, Mode 3, Mode 3B, Mode 3C, Mode 4 and Mode 5, in accordance of the adduct propagation in the stages of device within the pressure conditions prevailing.

According to an embodiment, the method can comprise finding similarities from the database with similar samples, as based on identified marker substances from the previous sample mass spectra in the database. In addition, in the embodiment variant of the method, the sample associated additional data is compared to similar associated additional data of the previous samples to find correlations from the additional data and the markers between the instant and previous gas samples. According to an embodiment, the comparison is made by the software packet as a system element. According to an embodiment variant the comparison comprises at least one of the following being used in it: an artificial intelligence algorithm to find patterns between the addition data and the marker substances, self-learning algorithm to assist the artificial intelligence algorithm, and neural network for optimization of the finding the marker substances.

According to an embodiment, database is in cloud, the system elements of the embodied MPCI MION system can constitute a network of systems contributing to the database to make it more rigorous. Individual terminals as system elements can access to the database via network. Edge computing (where the system is) will identify peaks in the chemical spectra, neural network can be embodied to be in the cloud in the main database, in suitable part, and according to an embodiment the cloud integrates the database with other data and provide analysis, for example meta data classification prediction, for example underlying condition corresponding to chemical composition of breath.

According to an embodiment, the MPCI-MION device is integrated to a mass spectrometer as its sample introduction device, so to form a MPCI-MION-mass spectrometer as a system variant of MPCI-MION system, to be combined with one or more embodiments of the present disclosure.

The invention also includes the following embodiments:
Embodiment 1. A Multi pressure chemical ionization multi-ion identification device, wherein said device comprises in a stack of ionization stages at least one atmospheric and/or high pressure ionization stage followed by at least one underpressure ionization stage in series following said at least one atmospheric and/or high pressure ionization stage in the device.
Embodiment 2. The Multi pressure chemical ionization multi-ion identification device according to embodiment 1, wherein, the MPCI-device further comprises a sample introduction port (100a) for sample with analyte molecules therein, said sample introduction port (100a) being followed by a stack (100b) of ionization stages (A, B, B′, C′, D′, E′), wherein said stack (100b) comprises at least one ionization stage in atmospheric or high pressure (A), (B) followed by a number of underpressure ionization stages (B′), (C′), (D′), (E′) in series of reducing operation pressure towards an exit to a mass spectrometer (MS) for a mass spectrum acquiring.
Embodiment 3. The multi pressure chemical ionization multi-ion identification device according to embodiment 1 or 2, wherein each ionization stages (A), (B), (B′), (C′), (D′), (E′) comprising a ionization-stage-specific number of ion injection towers (R1, R2,R3), (R4, R5,R6), (R7, R8, R9), (R10, R11,R12), (R13, R14,R15), (R16, R17, R18), each said ion injection tower being selected for a dedicated reagent (Rn), (Rm) emission in the ionized form at the entry to the corresponding ionization stage, for chemical ionization of analytes from the sample, in such a ionization stage in said ionization stage specific pressure lower than the high pressure stage pressure, and temperature condition.
Embodiment 4. The Multi pressure chemical ionization MPCI MION device according to embodiment 1, 2 or 3 wherein in each ionization stage with its plurality of reagent ion towers (R1, R2, R3) to provide dedicated ionization reagent ions each, said each ionization stages are positioned into a stack as stacked to planar geometry with planes (IR(A), IR(B), IR(B′), IR(C′), IR(D′), IR(E′),) each ionization stages as mutually parallel while perpendicular to sample flow entrance direction to ionization region of the Multi pressure chemical ionization multi-ion identification device (100),
Embodiment 5. The Multi pressure chemical ionization device (MPCI-MION device) according to embodiment 1, 2, 3 or 4 wherein each ionization stage has a predetermined thickness in the sample flow direction correspond the timescale of chemical ionization in the pressure and temperature conditions of the ionization stage to provide a stage specific reaction time for the ions emitted from the respective ion towers to chemically ionize analyte molecules in the sample, the so formed adducts being transported from the ionization region to next stage or to a mass spectrometer port for mass analysis of the species of the adducts.
Embodiment 6. The MPCI-MION device according to anyone of the previous embodiments, wherein each ionization reagent of the respective reagent ion injection tower in same plane (IR(A)) is configured to operate according to same ion production mechanism to provide respective reagent ions from a dedicated reagent ion tower.
Embodiment 7. The MPCI MION multi-ion identification device according to anyone of the previous embodiments, wherein the ionization mechanism of a reagent ion tower to ionize reagent molecules to reagent ion in the ion towers by the ionization mechanism of the ion tower is at least one of the following: X-ray, soft-X-ray, corona discharge, electrospray, xenon UV lamp, based ionization mechanism.
Embodiment 8. The multi-ion identification device according to anyone of the previous embodiments, wherein the polarity of a reagent ion tower produced reagent ions are adjustable to positive or negative ions.
Embodiment 9. The multi-ion identification device according to anyone of the previous embodiments, wherein at least one of the reagent ion towers comprises a filter to filter away multiply charged reagent agents away from entry to the ionization region.
Embodiment 10. The multi-ion identification device according to anyone of the previous embodiments, wherein the ionization region comprises a round cylindrical symmetry with a centerline (C) as a symmetry center of the stack (100b).
Embodiment 11. The multi-ion identification device according to anyone of the previous embodiments, wherein the reagent ion towers in the same stage) are aligned into a corresponding plane (IR(A), IR(B), IR(B′), IR(C′), IR(D′), IR(E′),) and have an off-set (D) to deflect from the direction of the center line (C) along the respective emitting lines).
Embodiment 12. A MPCI MION multi-ion identification system (Sys) comprising following items:
   at least one MPCI MION multi-ion identification device according to embodiments 1 to 11,
   at least one control unit to control the MPCI MION multi-ion identification system and its actuators for the operation in mass analysis of adducts formed from the constituents of the sample,
   a mass spectrometer (MS) to make said mass analysis,
   a database (DB) to store and process mass analysis results.
Embodiment 13. The multi-ion identification system of embodiment 12, wherein the system comprises a software packet (SW) configured to control operation of the MPCI MION multi-ion identification system.
Embodiment 14. The MPCI MION multi-ion identification system of embodiment 12 or 13, wherein the system comprises such a software packet (SW) that is configured to make group analysis to find and deduce marker substances from the results.
Embodiment 15. The MPCI MION multi-ion identification system of embodiment 14, wherein the software packet (SW) comprises at least one of the following: a machine learning algorithm, a neuron network solver for classification and optimization of data clusters, an artificial intelligence algorithm, such as penalized linear LARS, an elastic net regressions algorithm, random forests and recursive feature elimination algorithm, to be used to analyze, compare and predict chemical features of gaseous samples.
Embodiment 16. The MPCI MION system of any preceding clams 12 to 15, wherein the system comprises an ion detector to detect ions, said ion detector being configured to simultaneously utilize multiple selective ion chemistries both in negative and positive modes of detection.
Embodiment 17. The MPCI MION system of any one of the preceding embodiments 12 to 16, wherein the system is configured to detect extremely low vapor pressure, highly oxidized multifunctional organic molecules (HOM) from the sample.
Embodiment 18. A method identifying substances from a gas sample by using a multi-ion system (MPCI-system) of anyone of the previous embodiments 12 to 17, comprising:
   sampling a gas sample into a sample flow of the MPCI MION multi-ion identification device,
   allowing turbulence to decay to laminar flow conditions of the sample flow in a buffering region of the multi-ion identification device in the first stage,
   protecting the gas sample by at least one or two sheath flows at least in the buffering region,
   charging the gas sample constituents by reagent ion molecules formed for use in chemical ionization of said gas sample constituents to form adducts in corresponding stages with stage specific pressure and temperature,
   allowing the adduct to form from the gas sample constituents and reagent ion molecules in corresponding stages,
   leading the adducts to mass spectrometer for mass analysis,
   identifying the adducts and the gas sample constituents,
   storing to a database the identified gas sample constituents.
Embodiment 19. A mass spectrometer integrated together from the items according to any one of the embodiments 12 to 17 operable according to method embodiment 18.

The expression “a number of” refers herein to any positive integer starting from one (1), e.g. to one, two, or three.

The expression “a plurality of” refers herein to any positive integer starting from two (2), e.g. to two, three, or four.

The expression to comprise has been used as an open expression.

Different embodiments of the present invention are disclosed in the dependent claims.

### BRIEF DESCRIPTION OF THE RELATED DRAWINGS

Next embodiments of the present disclosure according to the invention are described in more detail with reference to the appended drawings in which

FIG. 1 illustrates an exemplary embodiment of an MPCI MION multi-ion identification device in accordance with the present disclosure of the invention, in a sampling state to be embodied with one or more embodiments of the invention, with two high pressure and/or atmospheric pressurized stages in the stack of the stages.

FIG. 1B illustrates the MPCI MION multi-ion identification device of FIG. 1 in an idle state, between consecutive sampling states of the device.

FIGS. 2 and 3 illustrate as examples of ion injection towers as reagent ion towers in layers of an embodied MPCI MION multi-ion identification device in accordance with the present disclosure of the invention. The atmospheric pressurized stages for operation as such can be used by one alone or both together in atmospheric pressure conditions, such being denoted also by NTP, to be embodied with one or more embodiments with low pressure stages such as in multi pressure chemical ionization MPCI MION directed embodiments.

FIG. 3A, FIG. 3B, FIG. 3C and FIG. 3D illustrate as an ensemble of examples of ion injection towers as reagent ion towers at ionization stages (also as stages for brevity) as layers of an embodied MPCI MION multi-ion identification device and its stages as low pressure units (LPU1, LPU2, LPUi, LPUi+1) in accordance with the present disclosure of the invention, for use in combination with at least one of the MPCI MION device's low pressure stages, with pressure ratios there between for two next successive stages as selected for embodied decimal fractions (P(A)/P(B), P(B)/P(B′), P(B′)/P(C′), P(C′)/P(D′) P(D′)/P(E′), for co-operation in the stack by one more MION stages (FIGS. 2 and 3, with respective pressures P(A), P(B)), as alone or together, in reduced pressure conditions, to be embodied with one or more MPCI MION-device related embodiments, to form a stack of the stages like the detail 100b in FIG. 1. In these examples, the stages for forming the stack 100b of the MPCI MION-device and system can be in reduced pressure or underpressure.

FIG. 4 illustrates an MPCI-MION device based MPCI MION system as an exemplary embodiment to use an MPCI MION multi-ion identification device in accordance with the present disclosure of the invention, to be embodied with one or more embodiments of the invention. According to the present disclosure the embodiment can control in addition to MION stages as such, also underpressure stages of MPCI MION system stages in FIG. 3A to FIG. 3D,

FIGS. 4A, 4B and 4C illustrate diversified controlling of MPCI MION device and the system, the stages of FIGS. 3A to 3D in accordance to the control functionalities explained with FIG. 4, with dedicated MPCI MION low pressure stages for the LPUs in FIGS. 3A to 3D, to be used in combination to one or more embodiments,

FIG. 5 is a diagram for an embodiment to group analytes for selection of reagents according to their basic-acidic properties and related functionalities to a number of groups for identification by use of an embodied MPCI MION multi-ion identification device,

FIG. 6 is illustrating an embodiment of the invention directed to a method to identify substances from a gas sample by using MPCI MION-based device of the MPCI MION-system,

FIG. 7 is illustrating schematically an embodied reagent ion tower structure as such,

FIG. 8 is illustrating a Multi-pressure Chemical Ionization (MPCI) device of a an embodied MPCI MION system as according to the present disclosure, with a number of stages as low pressure units in the stack 100b, based on MPCI MION-device and the system, to be used in combination of one or more embodiments,

FIG. 9 illustrates the MPCI MION-device of the system (FIG. 8) in Mode 1, to be used in combination to one or more embodiments of the present disclosure,

FIG. 10 illustrates the MPCI MION-device of the system (FIG. 8) in Mode 2, to be used in combination to one or more embodiments of the present disclosure,

FIG. 11 illustrates the MPCI MION-device of the system (FIG. 8) in Mode 3, to be used in combination to one or more embodiments of the present disclosure,

FIG. 12 illustrates the MPCI MION-device of the system (FIG. 8) in Mode 4, to be used in combination to one or more embodiments of the present disclosure,

FIG. 13 illustrates the MPCI MION-device of the system with a further underpressure stages (one in the example with an intermediate pressure range stage, 5-200 mbar) in the sections, i.e. with a number of stages in the stack 100b, to be used in combination to one or more embodiments of the present disclosure,

FIG. 14 is illustrative of a calibration of the MPCI MION-device of the system of FIG. 13, to be used in combination to one or more embodiments of the present disclosure,

FIG. 15 and FIG. 15A illustrate sample introduction alternatives to an embodied MPCI MION-device of the system, to be used in combination to one or more embodiments,

FIG. 16 illustrates calibration arrangement for the MPCI MION-device of the system, to be used in combination to one or more embodiments of the present disclosure,

FIG. 17 illustrates an example on Reagent production in reagent (ion) towers by a ion source assembly as embodied as such, to be used within a number of stages in the stack 100b, to be used in on ore more embodiment of the present disclosure,

FIGS. 17A, 17B and 17C illustrate examples on reagent vials with reagent chemicals as enclosed and/or impregnated in suitable part, for use according to FIG. 17, to be used in on ore more embodiment of the present disclosure,

FIG. 18 illustrates schematically use of ion source assembly for reagent ion injection to be used in on ore more embodiment of the present disclosure,

FIGS. 19, and 19A illustrate reagent molecule adduction in chemical ionization in presence of electromagnetic gradient, to be used in on ore more embodiment of the present disclosure,

FIG. 20 illustrates an MPCI MION-device and system based acquiring method, Method 1, to be used in the MPCI MION system, according to the present disclosure of the embodiments of the invention, to be used in combination of one or more embodiments, the method features as well as the related system elements and devices can be controlled by an embodied software package in combination to one or more embodiments,

FIG. 21 illustrate an alternative embodiment on the MPCI MION-device and system based acquiring method, Method 2, to be used in the MPCI MION system, according to the present disclosure of the embodiments of the invention, to be used in combination of one or more embodiments, the method features as well as the related system elements and devices can be controlled by an embodied software package in combination to one or more embodiments,

FIG. 22 illustrate a further alternative embodiment on the MPCI MION-device and system based-acquiring method, Method 2A, to be used in the MPCI MION system, according to the present disclosure of the embodiments of the invention, to be used in combination of one or more embodiments, the method features as well as the related system elements and devices can be controlled by an embodied software package in combination to one or more embodiments, and

FIG. 23 illustrate a further alternative embodiment on the MPCI MION-device and system based-acquiring method, Method 2B, according to the present disclosure of the embodiments of the invention, to be used in the MPCI MION system, to be used in combination of one or more embodiments, the method features as well as the system elements and devices can be controlled by an embodied software package in combination to one or more embodiments,

### DETAILED DESCRIPTION OF THE EMBODIMENTS

Same reference numerals in different figures (Figs) can be used to refer to similar objects, which do not necessarily be exactly identical, as a skilled person in the art understands from the embodiments of the invention.

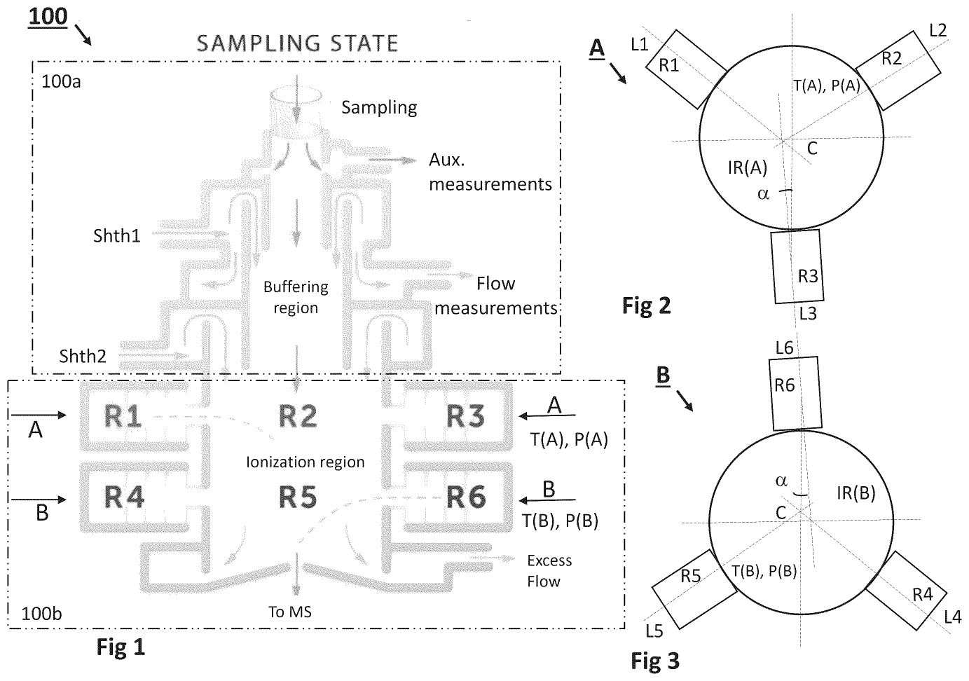

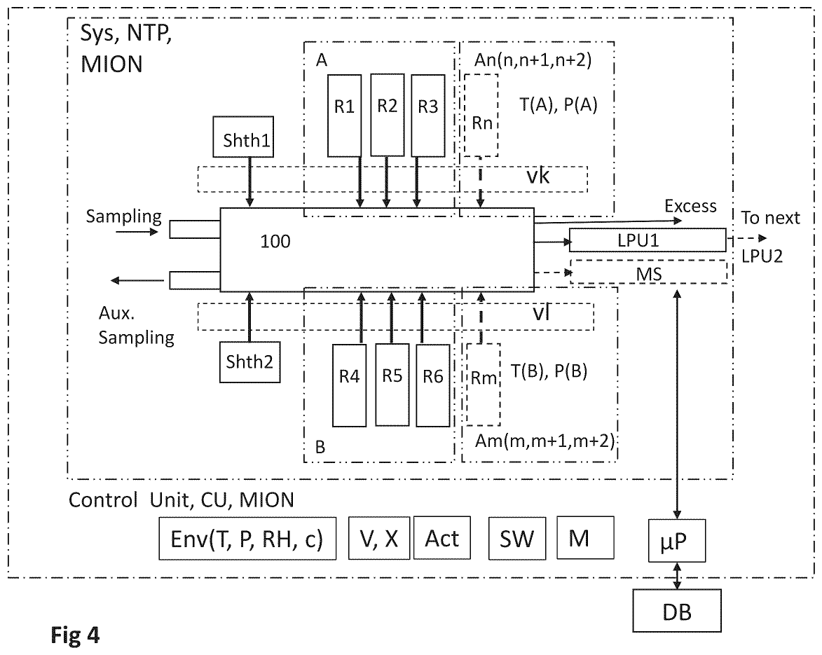

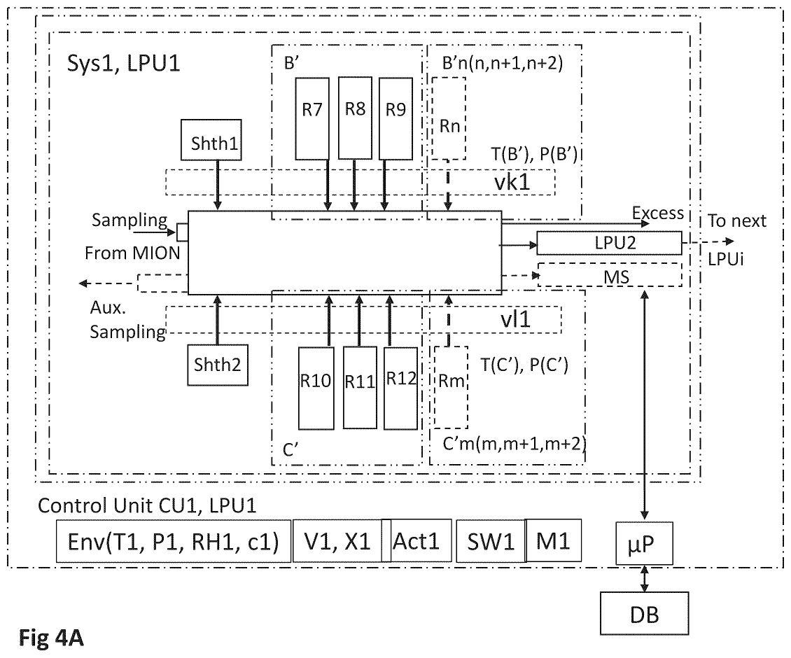

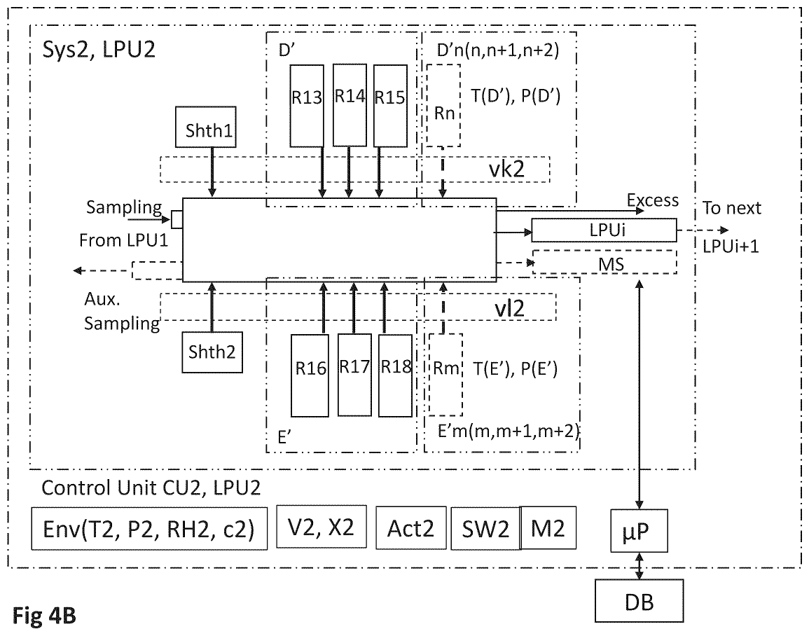

FIG. 1 illustrates multi-ion identification device 100 of a MION-system (Sys, FIG. 4), to be used with co-operation of a mass spectrometer MS (FIG. 4), in atmospheric pressures internal conditions as such.

It is notified that according to the present disclosure, Multi-Pressure Chemical Ionization (MPCI) system according to the present disclosure can comprise such a MION system as such as a part of the MPCI-system, although the MPCI-system has reduced pressure stages embodied as Low Pressure Units (LPUs) between the sample introduction and the mass spectrometer MS in the system.

According to an embodiment, in the MPCI-system including a multi-ion identification (MION) device 100, there is a buffering region 100a, in which the sheath flows enter to the multi-ion identification device, as well as the sample to the device. According to an embodiment, the buffering region can comprise a port to auxiliary measurements to further processing of the sample elsewhere and/or a redundant and/or a diverse analysis elsewhere, i.e. in a second device which can be similar, but is not necessarily the same.

The total flow can be measured at the port Flow measurement, as based on the direct measurement of the flow and/or the set flows in the other parts of the device according to the set up made by the control unit to control flow actuators in the system. The arrows in FIG. 1 illustrate flows, and the curved dashed lines paths of dedicated reagent ions from the corresponding reagent ion towers (R1, R6).

Reagent ion towers (R1, R2, R3, R4, R5, R6, Rn, Rm) can be embodied with acceleration and filtration of ions according to their charge. Reagent ion towers can charge reagent substances, being fed according to their dedicated feeds from the corresponding reagent substance sources, the feed and/or species being controlled by the control unit. The reagent ion towers, (also as ion towers) can utilize in the reagent charging soft x-rays, corona discharge and/or electrospray-mechanisms, to provide the reagents with wanted polarity to be used in the ionization region to chemically ionize sample molecules by the reagent molecules adducting to the sample molecules. Any charging mechanisms can be used, photon radiation, particle-based radiation as well as chemical ionization-based charging can be used in the MPCI-system in suitable part in the IMR-stages (Ion Molecular Reaction, a region where the chemical ionization is planned occurring) as well as in the API (Atmospheric Pressure Interface, as an interface of/to a mass spectrometer, where analyte ions enter the lower pressure compartments.) stages. Although name Atmospheric Pressure Interface used herein, it is not intended to limit the pressure only to the atmospheric pressures. A skilled person knows that also pressure range from atmospheric to pressures of 500 mBar, to 200 mBar or even down to 10 mBar can be used where applicable in the API called stages.

The sheath flows sheath1 and Sheath2 are arranged to follow the cylindrical geometry of the buffering region of the device, so that both sheath flows are introduced into the buffering region to form an annular sheath surrounding the sample flow, according to an embodiment the geometry and the flows being set so that the sheath flows and the sample flow has equal velocity, so to prevent unwanted vertical vortex mixing and/or turbulence formation. According to an embodiment, the flows are set to correspond laminar flow conditions by the control unit controlling the flow valves as flow actuators as system elements, under the surveillance of the software packet routines dedicated to flow maintenance.

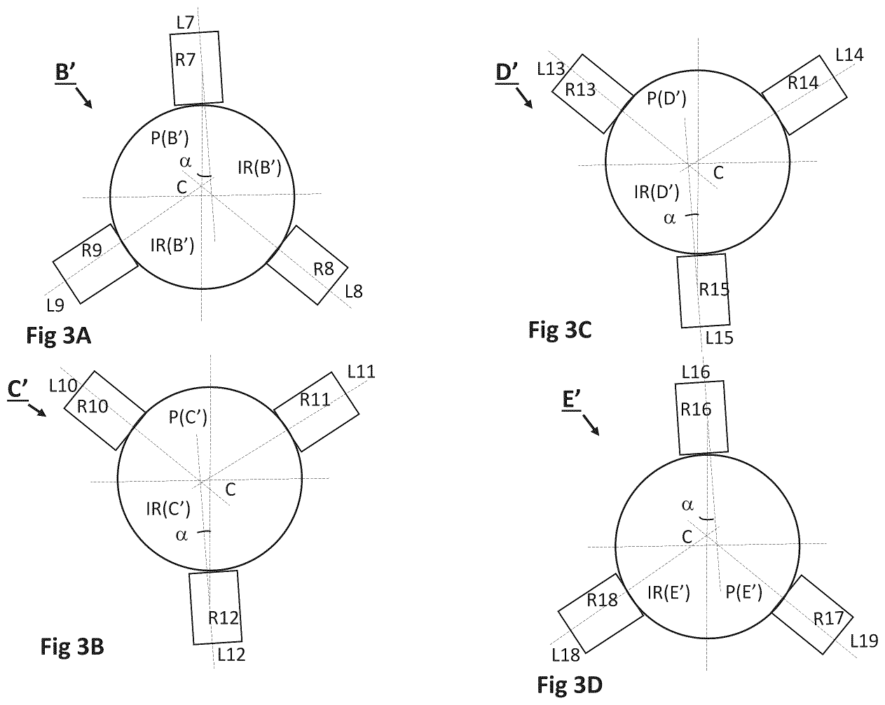

FIGS. 2 and 3 illustrate each a section forming a stack of the ionization regions (IR(A) and IR(B)) at the levels A and B. In FIG. 2, the capital C represents the geometric center line of the ionization region 100b at the level IR(A). The same geometric center axis line equals the geometric symmetry axis line of the buffering region 100a.

At the level IR(A), the lines L1, L2 and L3 represent the planar lining of the corresponding respective reagent ion towers R1, R2 and R3, so that R1 is lined along the L1 to inject ions to the direction indicated by the line L1. According to an embodiment of the invention of the present disclosure, the lines L1, L2 and L3 are misaligned (i.e. having an off-set) in a radial manner from the geometric center direction, by a sharp angle α.

According to an embodiment the angle is below 30°, according to an embodiment variant below 20°, according to a further variant below 10°, according to even further variant below 5°, but according to an embodiment variant between 0.7° to 35°

Similar geometry as at the level IR(A), at the level IR(B), the lines L4, L5 and L6 represent the planar lining of the corresponding respective reagent ion towers R4, R5 and R6, so that R4 is lined along the line L4 to inject ions to the direction indicated by the line L4. According to an embodiment of the invention of the present disclosure, the lines L4, L5 and L6 are radially misaligned (i.e. having an off-set) from the geometric center (capital C) direction, by a sharp angle α.

According to an embodiment the angle is below 30°, according to an embodiment variant below 20°, according to a further variant below 10°, according to even further variant below 5°, but according to an embodiment variant between 0.7° to 35°.

According to an embodiment, the misalignments selected to deflect between levels A and B, especially in such embodiments, in which the level R3 and R6 would be oppositely mounted to their respective own levels. According to an embodiment, such a misalignment is made on purpose to certain degree, to prevent the oppositely placed reagent ion towers (i.e. as exemplified to different levels the R3 and R6) to adversely affect each other's operation in the chemical ionization. According to an embodiment the off-set is made to same direction of rotation, so prevail a certain kind of equal sectors for the ionization probability with similar reaction times at the level, with equal efficiency to get the sample analyte to be charged by the reagent ions at the ionization region's sub-region.

According to an optional embodiment variant, the lines L1, L2 and L3 could be provided for such a mounting of R1, R2 and R3, so that lines L1, L2 and L3 would define a first conical mantel. However, such an embodiment would produce different ionization relaxation times to settle more easily to different parts of the ionization region than when planar. Such embodiment could be useful if such effect would be desired.

According to an optional embodiment variant, the lines L4, L5 and L6 could be provided for such a mounting of R4, R5 and R6, so that lines L4, L5 and L6 would define a second conical mantel. However, such an embodiment would produce different ionization relaxation times to settle more easily to different parts of the ionization region than when planar. Such embodiment could be useful if such effect would be desired. However, the first and second conical mantels as defining the reagent ion tower direction need not necessarily to be co-aligned.

In FIGS. 2 and 3 three angularly equally distributed injection towers as reagent ion towers are embodied in each level IR(A) and IR(B), as an example of the number and angular position of them. A skilled person realizes from these example embodiments, that the number of injection towers as reagent ion towers is not limited necessarily only to three per level, but can be varied to greater number, such as four, five or six according to the respective embodiments. The off-set can be embodied according to that what has been discussed with the R1, R2 and R3 concerning their off-sets. As skilled person in the art realizes also that the member of the levels (A, B) is not necessarily limited only the shown two, but can be according to the respective embodiment variants three, four or five, however, taking into account diffusion characteristics of the sheath gas material as well as the expected sample composition, to provide accordingly sufficient crosstalk suppression by the embodied number of reagent ion towers and purge time characteristics for each.

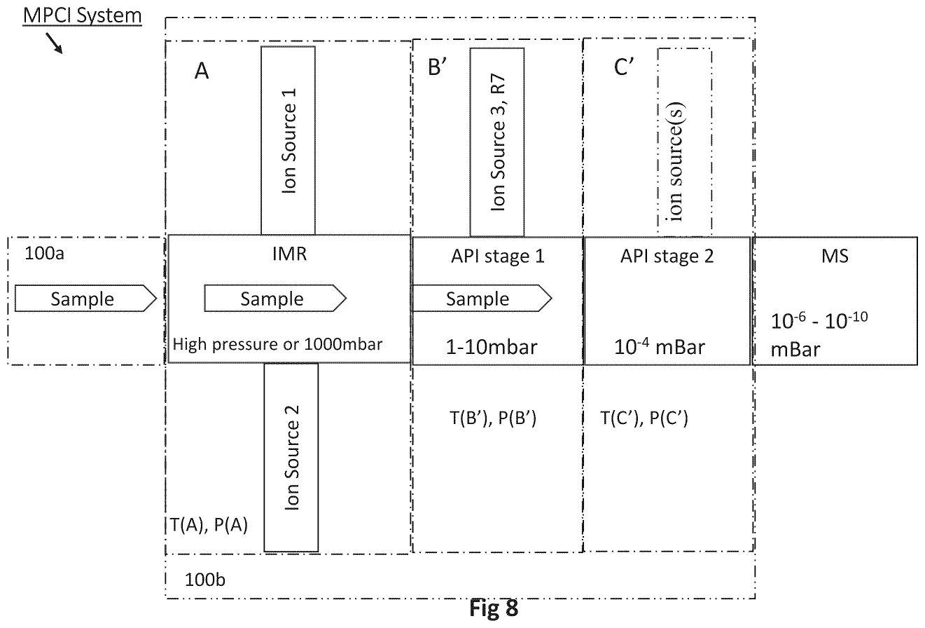

FIG. 3A illustrates a similar chemical ionization stage B′ with the ionization region IR(B′), as the one in FIG. 2, except that the B′ is operable in reduced pressure conditions (called also as Low-Pressure Unit 1, i.e. LPU1), for example such as disclosed in examples in FIGS. 8 to 12, FIG. 13 and FIG. 14, under the control of the corresponding control unit (CU1) illustrated for example in FIG. 4C. R7, R8, R9 are reagent ion towers as embodied for the reagents dedicated for use in that ionization region IR(B′), with the directed aim according to an embodiment either towards the centerline, or as deflected by angle α, either one embodied as being dependent on the turbulence generation importance in the used pressure conditions P(B′) in such stage. The directions are indicated by the respective markings L7, L8 and L9, following the respective numbering of ion tower numbering. Although three ion injection towers (R7, R8, R9) are shown, their number is not limited only to that in the example only.

FIG. 3B illustrates a similar chemical ionization stage as B′ with the ionization region IR(C′), as the one in FIG. 3A, except that the C′ is operable in a further reduced pressure conditions (called also as Low-Pressure Unit 2, i.e. LPU2), for example such as disclosed in examples in FIGS. 8 to 12, FIG. 13 and FIG. 14, under the control of the corresponding control unit (CU2) illustrated for example in FIG. 4C. R10, R11, R12 are reagent ion towers as embodied for the reagents dedicated for use in that ionization region IR(C′), with the directed aim according to an embodiment either towards the centerline, or as deflected by angle α, either one embodied as being dependent on the turbulence generation importance in the used pressure conditions P(C′) in such stage. The directions are indicated by the respective markings L10, L11 and L12, following the respective numbering of ion tower numbering. Although three ion injection towers (R10, R11, R12) are shown, their number is not limited only to that in the example only.

FIG. 3C illustrates a similar chemical ionization stage as D′ with the ionization region IR(D′), as the one in FIG. 3B, except that the D′ is operable in a further reduced pressure conditions (called also as Low-Pressure Unit 3, i.e. LPU3), for example such as disclosed in suitable part in examples in FIGS. 8 to 12, FIG. 13 and FIG. 14, under the control of the corresponding control unit (CU3) illustrated for example in FIG. 4C. R13, R14, R15 are reagent ion towers as embodied for the reagents dedicated for use in that ionization region IR(D′), with the directed aim according to an embodiment either towards the centerline, or as deflected by angle α, either one embodied as being dependent on the turbulence generation importance in the used pressure conditions P(D′) in such stage. The directions are indicated by the respective markings L13, L14 and L15, following the respective numbering of ion tower numbering. Although three ion injection towers (R13, R14, R15) are shown, their number is not limited only to that in the example only. FIG. 3D illustrates a similar chemical ionization stage as E′ with the ionization region IR(D′), as the one in FIG. 3C, except that the E′ is operable in a further reduced pressure conditions (called also as Low-Pressure Unit i, i.e. LPUi), for example such as disclosed in suitable part in examples in FIGS. 8 to 12, FIG. 13 and FIG. 14, under the control of the corresponding control unit (CUi) illustrated for example in FIG. 4C. R16, R17, R18 are reagent ion towers as embodied for the reagents dedicated for use in that ionization region IR(E′), with the directed aim according to an embodiment either towards the centerline, or as deflected by angle α, either one embodied as being dependent on the turbulence generation importance in the used pressure conditions P(E′) in such stage. The directions are indicated by the respective markings L16, L17 and L18, following the respective numbering of ion tower numbering. Although three ion injection towers (R16, R17, R18) are shown, their number is not limited only to that in the example only.

The indicated flows (Shth1, Shth2, Excess, as well as Flow measurement flow to a flow measuring device and the flow to the Aux. measurement) can be controlled by a Control unit (FIG. 4), under command of a software packet (SW, FIG. 4) for the MPCI-system including an embodied MION system (Sys, FIGS. 4, 4A, 4B, 4C), the software piece of the software packet being run by a dedicated microprocessor (μP, FIGS. 4, 4A, 4B, 4C) of the system to control the MPCI-system operations or parts thereof as system elements. There are embodied Shth1 as the sheath flow 1 and Shth2 as the sheath flow 2 to be used for sheathing the sample constituents travelling in the buffering region to enter the ionization region in the formed stack 100b, the sheath flows being controlled by the control unit (FIGS. 4, 4A, 4B, 4C) by using suitable ensemble of flow-dedicated actuators, so that for example an actuator to control Sheath flow 1 (Shth1) is dedicated to control the flow independently on the other flows as such, but however so that the set values to each flow under the control in whole represent a meaningful flow value being set by each flow-dedicated actuator, so that the sample flow is led through the multi-ion identification device 100 towards the mass spectrometer MS leading port. According to an embodiment, the sheath flows Shth1 and Shth 2 are used to protect the sample carrying flow through buffering region to ionization region.

According to an embodiment, the sheath flows are matched to laminar flow geometry to annularly surround the sample flow, so that the Shth1 surrounds the sample flow and the Shth2 surrounds the Shth1 and consequently also the sample flow at a distance. According to an embodiment the flow rate of the sample flow, sheath flow 1 and sheath flow 2 are set so, that they progress adjacently through the ionization region. These flows are set by the control unit (FIG. 4).

These actuators to be controlled as such as well as other actuators in the MPCI-system are illustrated in FIGS. 4, 4A, 4B, 4C by the actuator Act, referring a group of actuators for various purposes to maintain the operation of the system and its system elements.

Such actuators can be also used to control the operating environment of the device, to be used according to the ambient conditions, but also optionally to be operated in set conditions inside the device 100, by controlling the temperature (T), Pressure (P), relative humidity (RH) and/or composition (c) of the sheath gas of at least one of the sheath flow 1 and sheath flow 2. The controlling can be implemented by an ensemble of valves vk and/or another ensemble of valves vl, being illustrated in FIGS. 4, 4A, 4B, 4C in a schematic manner.

The way of drawing is illustrative also that the controlling by the control unit can be made to concern the reagent chemical inputs to the corresponding, arrow-indicated locations of the multi-ion identification device 100 in FIG. 1. Although polarity as such is not shown nor the selection, a skilled person knows from the disclosure of the embodiments that such can be made under the control of the control unit.

Although six reagent injection towers (R1, R2, R3, R4, R5, R6), also considered as reagent ion towers, are indicated in FIG. 1, where each injection tower can provide one or an ensemble of reagents in a ionized form to the ionization region, to combine with the analyte molecules from the sample, so to form adducts that are so chemically ionized, the number of the injection towers is not necessarily limited to the shown example. According to an embodiment, the polarity of each injection tower can be set individually by the control unit. According to an embodiment, a selection of user defined injection towers can be set to a certain polarity by the control unit, by using the user interface to command the actuators in the system to operate accordingly for the reagent chemical feed and/or polarity of the reagent ions.

Ionization of the reagents as such can be based on soft X-rays, corona discharge, or other suitable ionization mechanism as such to produce reagent ions for chemical ionization of the regent molecules, for combining with the analytes at the ionization region.

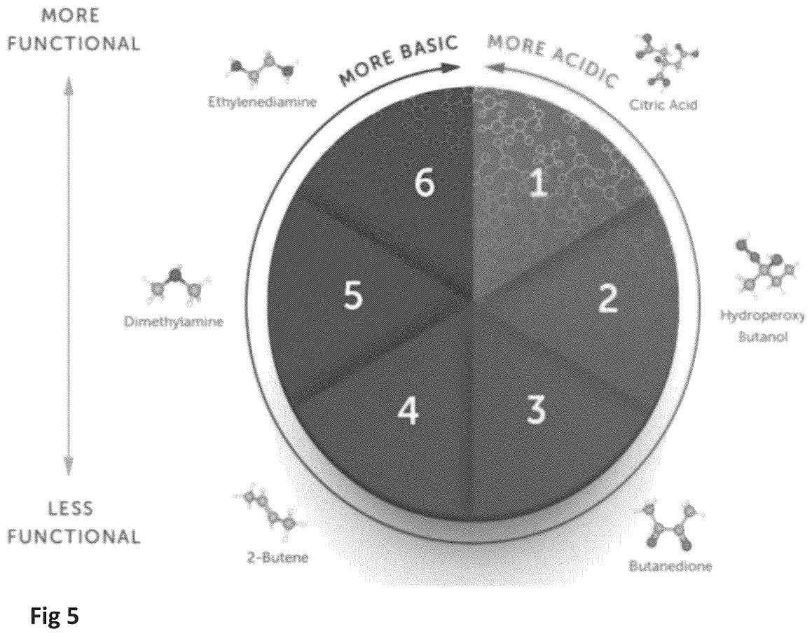

According to an embodiment variant, also further ionization injectors Rn, Rm can be used to provide similar ionization levels as indicated in FIG. 1 by the letters A and B at the corresponding levels in the stack for the ionization regions 100b of the MPCI-system element as device 100. Such levels are denoted by the expressions An(n, n+1, n+2), Am(m, m+1, m+2). At the An and Am, the letters n and m are used as arbitrary indexes to refer to a number of injectors. Accordingly, to the classification shown in FIG. 5, the number of classes can be defined accordingly for a finer classification of the analytes and the corresponding reagents.

In schematic FIGS. 4, 4A, 4B, 4C there is an actuator illustrative box with V, X markings, which refer to the control by the control unit. The control unit in FIGS. 4, 4A, 4B, 4C can control the voltages in embodiments, used in the MCPI-system by a suitable actuator being dedicated to the ionization for the reagents in each reagent injection port (R1, R2, R3, R4, R5, R6, Rn, Rm), via corona discharge, and/or X-ray tube voltage (X), but also to set the voltages for the mass spectrometer MS being controlled by the control unit, although the mass spectrometer had an independent control for its operations.

The controlling can be made according to the example in FIGS. 4, 4A, 4B, 4C by a dedicated microprocessor μP, running a controlling software, which can be embodied as a piece of software of the software packet SW. The software packet SW with the software codes for the routines are embodied as system element of the MION-system.

According to an embodiment such a software packet as the SW comprises means to constitute a database (DB, FIGS. 4, 4A, 4B, 4C) for the measurement results obtained from the mass spectrometer MS to identify the substances of a sample, and consequently the composition and abundance in the sample. Although being drawn apart from the dashed lines, the database can also be or have a part in the system's permanent part memory M.

The way of drawing the control unit and the system as the embodied MPCI-system, is selected to indicate that the control unit is controlling the MPCI-system. However, the control unit is considered as a part of the system as a MPCI-system element, even if the controlling were diversified to concern certain parts of the MPCI-system. The location as well as the implementation can be embodied in several ways. However, according to an embodiment the control unit(s) can be in suitable part also diversified so that some of the actuators are at the MPCI-system in its multi-ion identification device, and for example the microprocessor within the mass spectrometer, or even in a network location so facilitating remote control of the MPCI-system and its elements.

According to an embodiment such a software packet SW can comprise signal processing tools to analyze the mass spectra of the mass spectrometer MS, but according to an embodiment variant also tools for cluster/group analysis, as well as for correlation calculations to find marker substances from the samples. The software package can also have machine-learning algorithm to educate the MPCI-system in the mass analysis and the acquiring of the spectra, to find certain substances and their interrelations in the spectra as acquired and/or in the database. In other words, the software packet as a software suite consists of software services related to hardware control, mass spectrometer data analysis and user interface. The software enables use of the system with ease for the operator, as well as ensures good and consistent data quality. In addition, the software packet will perform for completing the tasks with various levels of required automation of the sampling interface, including potential external system elements such as thermal desorber, robotic manipulators etc., MPCI-system inlet also for parts for the MION-inlet, mass spectrometer and data processing pipeline, to a high degree. The output of the data processing software can comprise peak lists (sets of (m/z, intensity)-pairs), to be further analyzed together with the auxiliary data, as a part of the knowledgebase. A simple dedicated user interface is embodied to guide to take a sample through the sampling process and ensure the data quality. Machine learning can educate the MPCI-system to perform sequences in an automated manner, according to the resemblance to previous tasks, as being recorded and sequences therein recognized suitable to similar acquisition.

According to an embodiment of the present disclosure, FIG. 1 illustrates a concept of how six MION part sources (reagent ion towers) can be used in parallel switching between reagent ions, for an atmospheric pressure conditions in use. The switching happens in less than 1 second and can prevent neutral reagent entry to the flow stream.

FIG. 4C is illustrating controlling of dedicated Low Pressure Units LPU1, LPU2, LPUi and LPUi+1, such as the stages B′, C′, D′ and E′ as discussed within the examples relating to respective FIGS. 3A to 3D, but without an intention to limit only to the shown number of the stages, which is considered by the markings LPUi and LPUi+1 with the corresponding control units CUi and Cui+1. The controlling units in FIG. 4C can be physically diversified, or according to an embodiment implemented by a software into the computer's microprocessor's (uP) memory. Such diversification can be also obtained by using diversification according to a number of the microprocessor's cores in applicable extent.

FIG. 5 is a diagram for an embodiment to group analytes (i.e. target molecules with examples of such) for selection of reagents according to their basic-acidic properties and related functionalities to a number of groups for identification by use of an embodied multi-ion identification device.

In the example of FIG. 5, the target molecules are divided into six groups based on their chemical composition. The multi-scheme inlet developed here can cover practically all of these groups in a semi-continuous manner.

In order to target virtually every gas-phase chemical compound, there is a need for a collection of ionization reagents. With increasing functionalization of a molecule, simple molecular parameters become less well defined. A good simple example is offered by amino acids, which have characteristically both acidic and basic functionalities, with the acid-base behavior changing depending on the structure of the rest of the molecule. Thus, here we adopt an arbitrary, yet in a sense a more chemically meaningful definition, in which the molecules are labelled based on their functional group composition—and thus also on their ionization characteristics.

For such an approach variant in accordance of the present disclosure, the target molecules are divided into six groups that range from acidic, highly-oxidized and highly functionalized best detected with an adduct forming Negative Polarity Chemical Ionization (aNPCI) reagent ion (group 1), through reduced, naked hydrocarbons for which the best sensitivity is obtained by well-chosen H-transfer reagents (group 3 and 4), and finalizing again into highly functionalized, and thus also highly-oxidized, but rather basic compounds best detected with an adduct forming Positive Polarity Chemical Ionization (aPPCI) method (group 6). The biggest differences between groups 1 and 6 are the specific oxidized substituents and the nature of hydrogen bonding interactions they provide (i.e., in group 1H-bond donors, whereas in group 6H-bond acceptors). As illustrated, MION type of a multi-scheme inlet is able to cover all of these groups with carefully selected reagent ion combinations, which will be briefly explained next.

Group 1 reagents work almost solely with an aNPCI mode at atmospheric pressure, and the prototype reagents used here are nitrate ion (NO3-) and halogens (I—, Br—), about which the authors of the outstanding document have an extensive previous experience with. The main targets in group 1 are the most acidic and most functionalized molecules, which generally have very low gas-phase concentrations, and thus extreme selectivity and sensitivity offered by these aNPCI reagents are required. Similar characteristics are found from the group 6 target compounds (mainly the small gas-phase concentration and strong surface activity) with the important difference of being either at most only slightly acidic, or even basic. The group 6 compounds are thus best detected with an aPPCI approach (e.g., adduct formation with certain complex amine derived reagent ions). Groups 2 and 5 contain the moderately functionalized targets, which generally can sustain considerably higher gas-phase concentrations, and thus less sensitive (and selective) method is required for their quantification. An example of an aNPCI for group 2 is a carboxylic acid derived reagent ion, whereas for group 5 simple amine derived reagents are likely to work well. For the remaining least functionalized groups 3 and 4, a collection of H-transfer reagents is applied, and are formed, for example, from simple ketones and alcohols. The abovementioned reagent ions for groups 1 to 6 serve as an important example, but such grouping is not necessarily limited only to the shown instant example, which is also embodied and indicated in FIG. 4 by the optional additional injection layers illustrated by the injection layers An and Am. to be applied.

The reagent selection to correspond to the grouping can be set at the initial set up of the system and/or in an update of the system.

According to an embodiment an acquiring method (600), using such an embodied MPCI-MION system comprises:
sampling (601) a (gas) sample into a sample flow of the MPCI-system for the multi-ion identification by the devices as the system elements,
allowing (602) turbulence to decay to laminar flow conditions of the sample flow in a buffering region of the MPCI-multi-ion identification device,
protecting (603) the (gas) sample by at least one or two sheath flows at least in the buffering region, at least at the atmospheric pressure conditions,
charging (604) the (gas) sample constituents by reagent ion molecules formed for use in chemical ionization of said gas sample constituents to form adducts, in the respective pressure conditions in the stages of the MPCI MION system,
allowing (605) the adduct to form from the gas sample constituents and reagent ion molecules,
leading (606) the adducts to mass spectrometer for mass analysis,
identifying (607) the adducts and the gas sample constituents by a software packet routine for mass analysis,
storing (608) to a database the identification information of the gas sample constituents.

According to an embodiment, the method can comprise finding similarities from the database with similar samples, as based on identified marker substances from the previous sample mass spectra in the database, in which machine learning can help. In addition, in the embodiment variant of the method, the sample associated additional data is compared to similar associated additional data of the previous samples to find correlations from the additional data and the markers between the instant and previous gas samples. According to an embodiment, the comparison is made by the software packet as a system element, so comprising a machine learning package to do the comparing. According to an embodiment variant the comparison comprises at least one of the following being used in it: an artificial intelligence (AI) algorithm to find patterns between the addition data and the marker substances, self-machine-learning algorithm to assist the artificial intelligence algorithm, and neural network for optimization of the finding the marker substances.

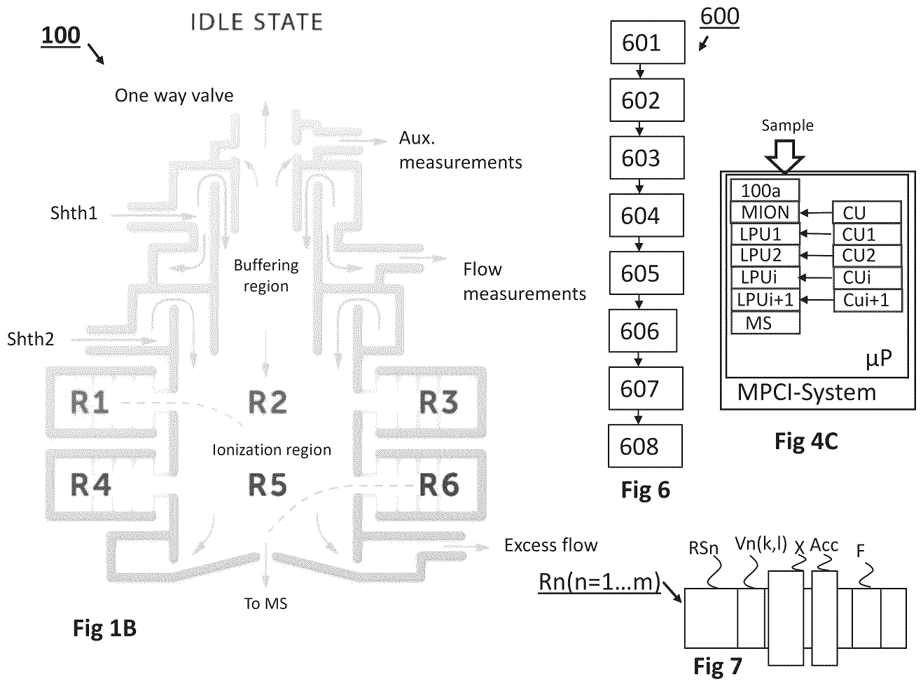

FIG. 7 illustrates schematically an embodied reagent ion tower structure. The reagent ion tower in question can be R1, R2, R3, R4, R5, R6, Rn, Rm, which is illustrated by the expression Rn(n=1 . . . m). RSn denotes to a dedicated source of reagent, vₙ(k,l) denotes to a dedicated valve to control the reagent RSn feed, X denotes to charger, independently is the charger embodied by soft X-rays, corona discharge, electrospray or a combination thereof. The polarity can be changed between negative positive and neutral according to the control unit settings. Acc denotes to an accelerator of charged reagent ions, which can be implemented by electric fields. In addition, the reagent ion tower can comprise also a filter F, to filtrate unwanted polarity and/or charge carrying ions away from the reagent ion tower output. According to an optional embodiment, the electric filter can be embodied within the accelerator Acc as integrated.

Consequently, a skilled person may, on the basis of this disclosure and general knowledge, apply the provided teachings in order to implement the scope of the present invention as defined by the appended claims in each particular use case with necessary modifications, deletions, and additions.

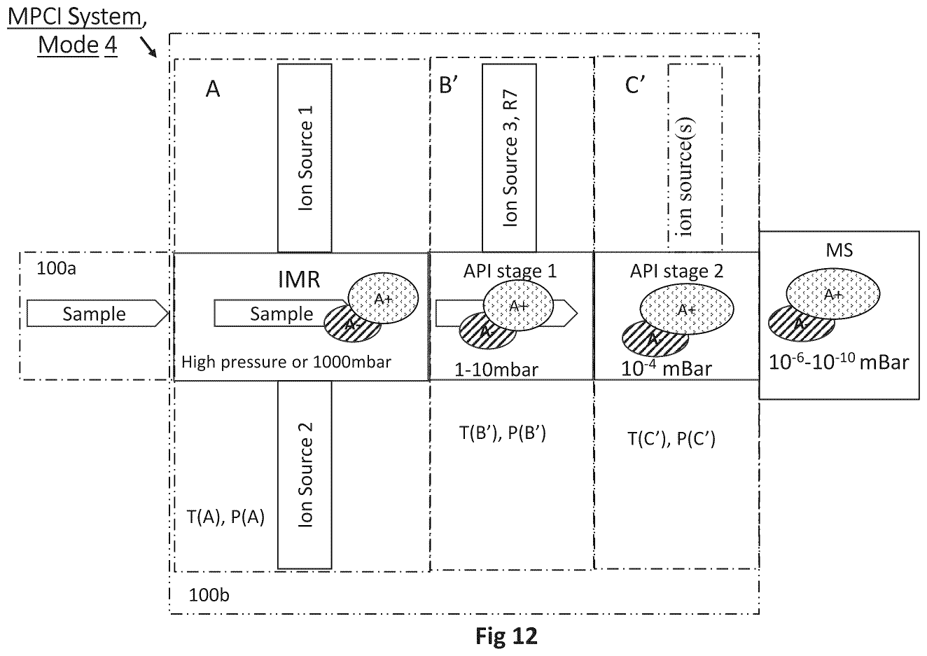

FIG. 8 (Slide 3) shows a Multi-Pressure Chemical Ionization (MPCI) system as a set up with different pressure conditions with shown stages in the stack 100b, for sampling and acquiring mass spectra by the mass spectrometer MS. The schematic diagram can be implemented in accordance to differently pressurized conditions of the stages cited also levels as such, in a stepwise scheme of the stages (IMR, API1, API2, API3) from the IMR towards the mass spectrometer MS as indicated in the FIG. 8 and related further Figs with similar notation, but different pressurizations as embodied in such examples (Slides). However, such an implementation of pressure conditions as well as temperature conditions can be made in accordance with such a disclosure as indicated in FIG. 4 about a controller, as addressed to control the stages of the system stages individually, the stages being isolated from each other except the sample propagation channel in FIG. 8. Accordingly, with such a provisions that as the IMR stage in FIG. 8 corresponds the stage A in the FIGS. 1, 2 and 4, also in a similar manner for the stepwise pressure scheme, to have pressure controlled (cf, by the controller in FIG. 4) as further stages added so that for example in FIG. 2 the stage A has been controlled to pressure conditions of FIG. 8 (Slide 3) IMR to around 1000 mBar, the consequential API1 stage, as corresponding to the stage B in said FIGS. 1, 3 and 4, but in FIG. 8 in pressure being selected to 1-10 mBar for that stage thus denoted as B′ (FIG. 3A). Alternatively an intermediate stage with pressure in 10-200 mBar range. Further similarly oriented stages in series as the A and B, can be added to follow in a similar way in series as shown in FIG. 8 such as API stage 2, but as have been controlled to pressure condition according to the FIG. 8, the API stage 2 is in the example in a pressure conditions of 10⁻⁴ mBar, and the mass spectrometer MS in the prevailing pressure conditions of 10⁻⁶-10⁻¹⁰ mBar therein. The layer C′ (FIG. 3B) is a similar layer as layer B′ (FIG. 3A), but provided with the dedicated pressure conditions as disclosed by the numeric non-limiting example, not limit only to the shown exemplary pressure values. Although mere indication about a presence of an ion sources potential is shown to correspond the reagent sources as ion regent towers, a skilled person in the art knows from the embodiments that the stage/layer C′ can have one or more reagent towers, if applicable for the mass analysis to be made. In similarly embodied is the pressures also in conditions of FIG. 12 (Slide 7) for the API2, and MS stages, as exemplified in Figs for some embodiments. The API stages/layers can have as well as the IMR applicable ion optics and vacuum system of the Massa spectrometer MS.

In FIG. 8-12, the mass spectrometer MS in FIG. 8 embodiment examples can be Time of Flight mass spectrometer (TOF), Fourier Transform-type (FTMS), or Quadrupole-type (QPMS) mass spectrometer.

The control as such is considered being implemented as in example of the FIG. 4, for the whole internal pressure control, but contrary to a previous MION related embodiments as such in atmospheric pressure, in the FIG. 8 and further Figs (slides) as embodied in a stepwise manner in reducing pressure conditions of the stages/levels accordingly so being controlled by the controller to the respective pressure level as such. The reagent ions in the API stages are not limited only to the disclosure in the named Figs (Slides), but different ions can be selected according to ion sources, for example as in the indications of FIGS. 18 and/or 19 (slides 13 and/or 14).

In some embodiments, also other ion production methods and devices related thereto can be used where applicable, even in some embodiments as based on known as such about ion injection.

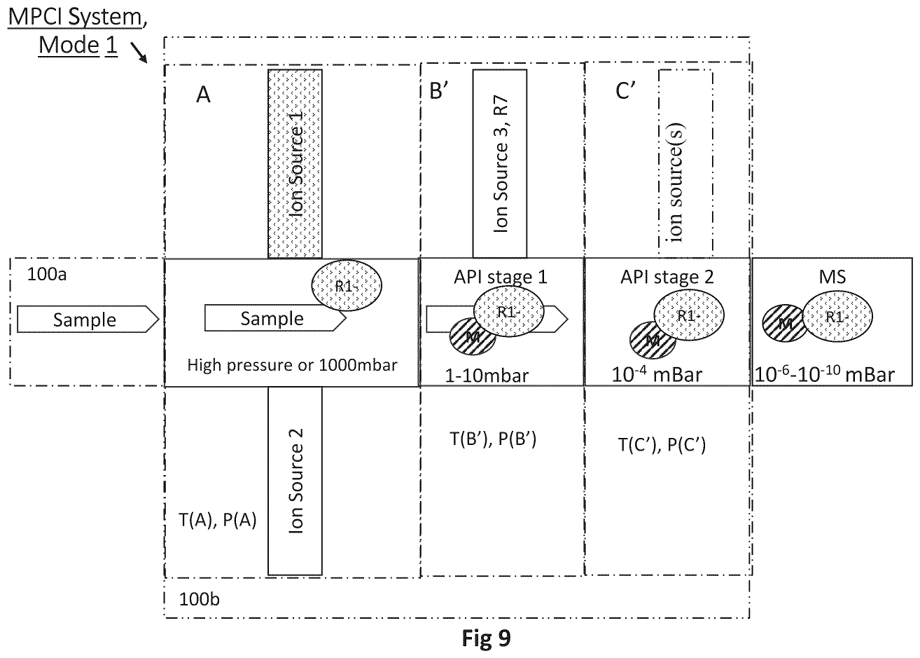

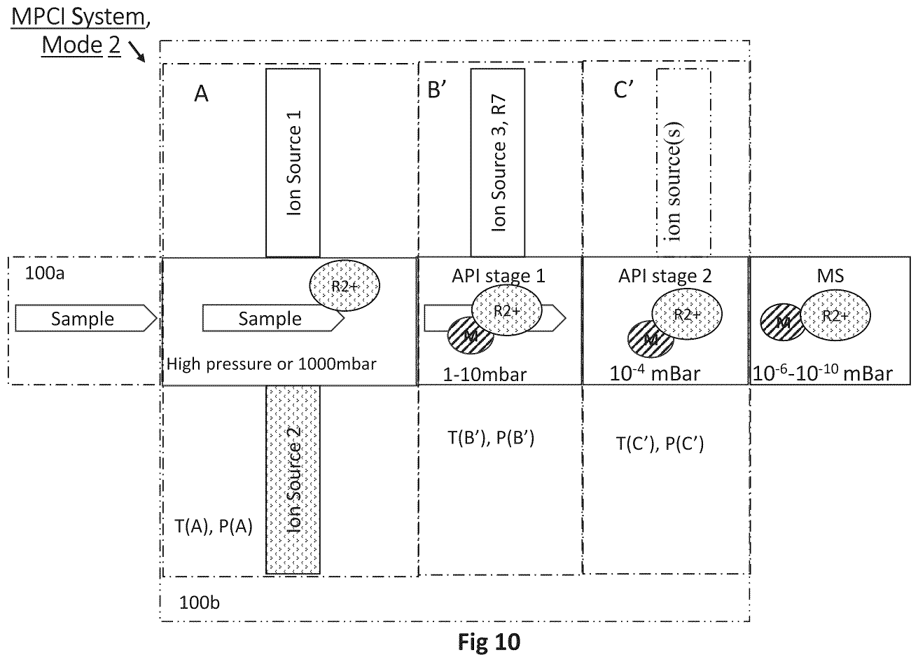

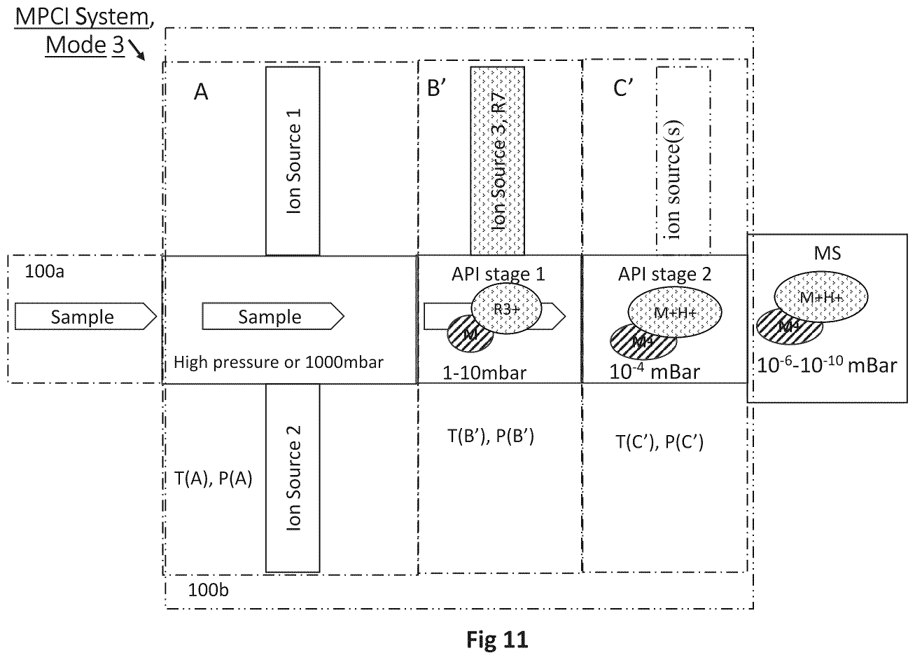

In FIG. 9 (slide 4), FIG. 10 (Slide 5) and FIG. 11 (Slide 6), the sample propagation has been considered according to the disclosure of said Figs (slides), in synergy with the apparatus disclosures of the present disclosure in FIG. 2 to FIG. 4. The Figs (slides) disclose also several modes Mode 1, Mode 2 and Mode 3 respectively, as indicated by these acronyms accordingly within the sample route and condition under the control of the controller in FIG. 4, to the mass spectrometer MS.

In FIG. 9 (slide 4), the Multi-Pressure Chemical Ionization MPCI MION system, according to the present disclosure is illustrated in the example as being in Mode 1. Sample is introduced to the IMR (as in FIGS. 1 and 2 via the 100a part to level A. The sample is comprising analyte molecules (M), from which an ensemble of analyte molecules (M) as present are addressed to detection in mass spectrometry (MS), to travel through the stages API 1 and API 2, in the controlled (Controller in FIG. 4) respective pressures (and temperatures) according to the selection, The temperature can be also controlled by the controller.

In FIG. 9 the indicated ion source 1 (i.e. similar to reagent source R1 for example, FIG. 2) is activated, and the rest reagent ion sources Ion source 2 and Ion source 3 are off, (i.e. corresponding the reagent sources R2, and R4, respectively as in FIGS. 2 and 3).

Reagent R1 in the FIG. 9 example is selected to be used as an anion (with − polarity), although alternatively also positive polarity (cation with + polarity) could have been set, if so wanted for the mass analysis goals.

For the example in FIG. 9, there was used an adduct formation in the Mode 1, However, also proton transfer as such and charge transfer (electron capture) reactions might happen resulting in M+H+, M−H−,or M−, M+ ions.

In the example, the Reagent 1 (R1) from the ion source 1 can be selected as Nitric acid (NO₃—), Dibromomethane (Br—), AceticAcid (AA-), as examples to mention some reagent substances in their ion form. However, it is advantageous to selected to Reagent 1 R1 such reagent that advantageously ionizes acidic and electronegative species, including HOMs—Highly Qxygenated Molecules, ULVOCs and SVOCs and such in the Mode 1.

In FIG. 9 system, in synergy to the embodiment in FIG. 2, the adduct formed by the analyte M and reagent R1-travels through the stages and continues to an additional (additional to the stage B′ in FIG. 3A) API stage 2 (level C′, FIG. 3B) in the pressure conditions of 10⁻⁴ mBar, to finally end up to the mass spectrometer MS in the prevailing pressure conditions of 10⁻⁶-10⁻¹⁰ mBar therein.

As in FIG. 8, for the layers A, B′ and C′ (stages IMR, API 1 and API2, respectively, also as respective levels, according to the pressure levels), also in FIG. 9, there is shown an option for the layer C′ as a similar layer as layer B′, but provided with the dedicated pressure conditions as disclosed in FIG. 9 conditions, as the pressure in layer B′ being marked by P(B′) and for layer C′ by P(C′). Similar way, also temperatures T(B′) for layer B′ ( ) and for layer C′ by T(C′) have been indicated. These conditions (T,P) in FIG. 9 do not need to be necessarily the same as in FIG. 8, despite of similar symbols used for the dedicated conditions in FIG. 8.

Although mere indication about a potential presence of an ion sources is shown to correspond the reagent sources as ion regent towers, a skilled person in the art knows from the embodiments that the layer C′ can have one or more reagent towers, for the reagents in ionized form, if applicable for the mass analysis to be made.

In FIG. 10 (slide 5), the Multi-Pressure Chemical Ionization MPCI MION system according to the present disclosure is illustrated in the example as being in Mode 2. Sample is introduced to the IMR (as in FIGS. 1 and 2 to level A). The sample is comprising analyte molecules (M), from which an ensemble of analyte molecules (M) as present are addressed to detection in mass spectrometry (MS), to travel through the API stages 1 and 2, in the controlled (Controller in FIG. 4) respective pressures according to the selection in the mentioned API stages, The temperature can be also controlled by the controller, same or similar to that in FIG. 4.

In FIG. 10 the indicated ion source 2 (i.e. similar to reagent source R2 for example, FIG. 2) is activated, and the rest reagent ion sources Ion source 1 and Ion source 3 are off, (i.e. corresponding the reagent sources R1, and R3, respectively as in FIG. 2).

R2 in the FIG. 10 example is selected to be a cation (with + polarity), although alternatively also negative polarity (anion with − polarity) could have been set, if so wanted for the mass analysis goals with such substances in such form and mode.

For the example in FIG. 10, there was used an analyte M and reagent R2 in adduct formation in the Mode 2, However, also proton transfer as such and charge transfer reactions might happen resulting in M+H+, M−H−,or M−, M+ ions.

In the example, the Reagent 2 (R2) from the ion source 2 can be selected as Acetonilacetone (AcAc+), Diethilamine (DEA+), Sodium (Na+), Acetone (Ac+), as examples to mention some reagent substances. However, it is advantageous to selected to Reagent 2 R2 such reagent that advantageously ionizes basic compounds such as amines, lower oxygen containing compounds SVOCs, VOCs, peroxides, ketones.

In FIG. 10 system in synergy to the embodiment in FIGS. 2, 3, 3A and 3B, the adduct travels through the stages that were illustrated, and continues to an additional stage/pressure level C′ (additional to the stage B′ in FIG. 3A) the API stage 2 being in the pressure conditions of 10⁻⁴ mBar, to finally end up to the mass spectrometer MS in the prevailing pressure conditions of 10⁻⁶-10⁻¹⁰ mBar therein.

As in FIG. 8, for the layers A, B‘ and C’, also in FIG. 10, there is shown an option for the layer C′ (FIG. 3B) as a similar layer as layer B′ (FIG. 3A), but provided with the dedicated pressure conditions as disclosed in FIG. 10 conditions, as the pressure in layer B′ being marked by P(B′) and for layer C′ by P(C′). Similar way, also temperatures T(B′) for layer B′ and for layer C′ by T(C′) have been indicated. These conditions (T,B) in FIG. 10 do not need to be necessarily the same as in previous FIGS. 8 and/or 9, despite of similar symbols used for the dedicated conditions in these Figs.

Although mere indication about a presence of an ion sources potential is shown to correspond the reagent sources as ion regent towers, a skilled person in the art knows from the embodiments that the layer C′ (FIG. 3B) can have one or more reagent towers, if applicable for the mass analysis to be made.

In FIG. 11 (slide 6), the Multi-Pressure Chemical Ionization MPCI MION system according to the present disclosure is illustrated in the example as being in Mode 3. Sample is introduced to the IMR level as in FIG. 10, via the part 100a, for example to the prevailing pressure of 1000 mBar. The ion sources 1 and 2, (respective ion towers 1 and 2 are off). The regent ion source 3 (i.e. R7 in FIG. 3A) as such is active to provide the reagent substance R3. Although selection of cation or anion is possible, a cation was selected in this example, to provide chemical ionization to the analyte molecule M, to form an adduct.

This can result in adduct formation as such, but also proton transfer and charge transfer reactions might happen resulting in M+H+, M−H−,or M−, M+ ions.

In this example embodiment the Ion Source 3 is located inside the first stages of the MS API interface (API1), where the pressure drops to pressure level of 1-10 mBar. At this point number of collisions goes down and energy of such goes up allowing for efficient PTR (Proton Transference Rate) and charge transfer ionization of non-polar compounds and compounds with low proton affinity.

Accordingly the Reagent 3 can be embodied as: Water (H30+), Ammonia (NH4+), PAH (pyrene or fluorenthene—creating radical ions), or Methane (CH5+ or CH3+), etc. (similarly suitable substance in same kind of formations).

Accordingly Reagent 3 can advantageously be an efficient PTR or charge transfer reagent to ionize non-polar compounds, simple ketones, VOCs, PAHs etc.

In FIG. 12 (slide 7), the Multi-Pressure Chemical Ionization MPCI MION system according to the present disclosure is illustrated in the example as being in Mode 4. In this mode all of the sources are off and the instrument can measure ambient ions, but another option is to perform zeroing (acquiring background spectrum to check the system for noise). The ions A(+/−) illustrate ambient ions.

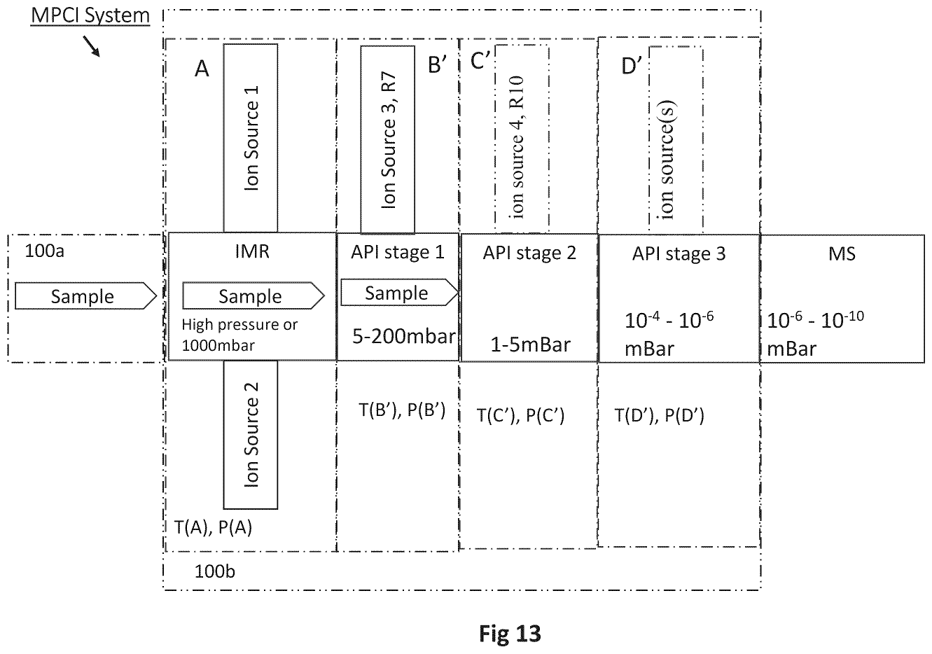

In FIG. 13 an alternative embodiment has been illustrated, with an extra API 3 stage/layer. The pressure levels in the API 1, API2 and API3 are set in the alternative embodiment variant respectively as 5-200 mbar in API1, 1-5 mBar in API 2 and 10⁻⁴-10⁻⁶ mBar for the API 3 level/stage. The mass spectrometer MS is in the embodiment in pressure level of 10⁻⁶-10⁻¹⁰ mBar. Alternatively a system with vacuum Compartments to even more gradually reduce the pressure levels and one more source to ionize analyte at different pressure and residence time. A skilled person in the art knows, that the division of pressure levels between the IMR and MS can be controlled by FIG. 4-type controller in a different way without intention to limit only to the shown example. IMR has been considered also here similarly as in FIGS. 8 to 12 as an ion-molecular reactor, API as atmospheric pressure interface (ion optics and vacuum system of the mass spectrometer). The MS mass spectrometer can be selected as a Time Of Flight, Fourier Transform, Iontrap, Quadrupole, or other suitable mass spectrometer to operate in such pressure conditions.

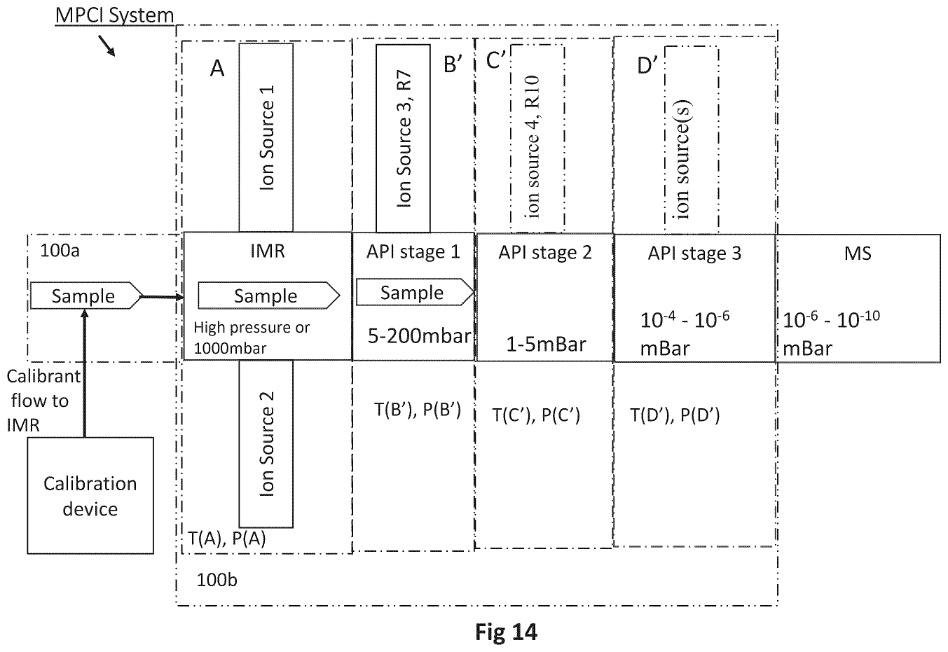

In FIG. 14 (slide 9) a further alternative embodiment has been illustrated, in accordance to the FIG. 13 embodiment, but the system embodied with a calibration device, to provide relatively stable feed of a known compound to provide overall calibration of the system.

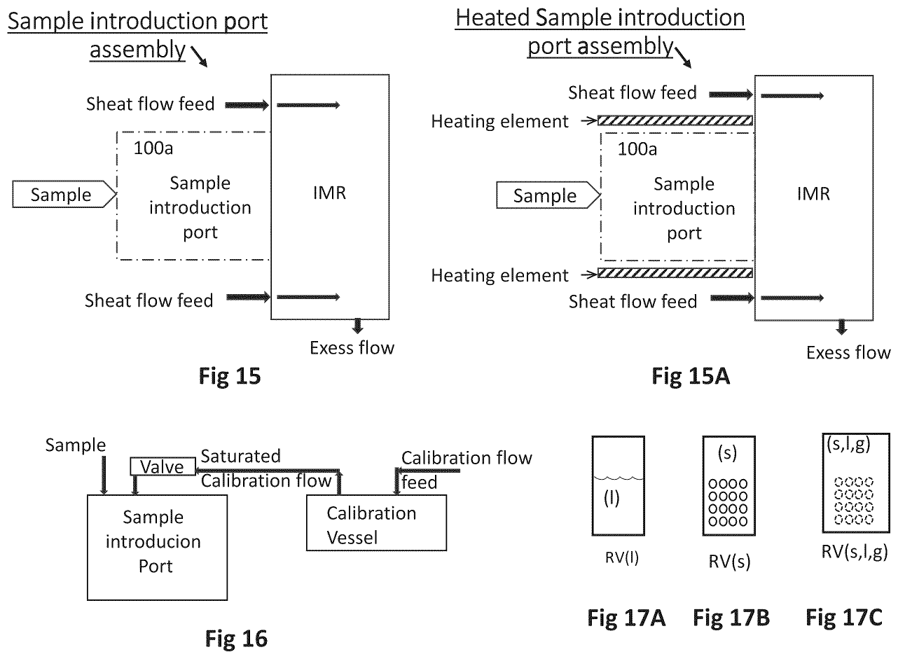

FIG. 15 (slide 10) is illustrating a sample introduction port assembly, similar to the part 100a in MION devices (FIG. 1) in applicable part. Such a sample embodied introduction port can be used in embodiment examples illustrated in FIGS. 8 to 14.

According to the embodiment, the Sample introduction port is comprising a tube, advantageously made of smooth material to provide a laminar flow (e.g. electropolished stainless steel, in avoidance of unnecessary turbulence). Diameter and length of such a tube should match flow rate to achieve most optimal flow profile and so to avoid turbulence generation.

In the assembly, the Sheath flow assembly is arranged to wrap the sample flow centerline inside the IMR and reduce non-wanted memory effects and provide a clean gas buffer in the ionization region.

Alternative use to create backpressure in the IMR by increasing the sheath flow flowrate and restricting excess flow vacuum to prevent sample entering the IMR, thus creating a “zero state” of clean gas to measure instrument's chemical background.

As illustrated in the example in FIG. 15A (slide 11), the sample introduction port indicated in FIG. 15 can be provided by a heating element. According to a further variant, such a heating element can be accompanied by a cooler, such as a Peltier element, to provide a control to the sample temperature, to be controlled according to a controller similar as shown in FIG. 4 and/or FIG. 4C.

Heating element implementation of the sample introduction port can comprise such a heating element (such as inductive heating elements, ceramic heaters, infrared heaters) arranged to heat the sample introduction port, preferably in an uniform way in the direction of the sample propagation. This is done to reduce memory effects of the sample introduction port, and by heating it to high temperature to clean the surfaces of the port from contamination, when such a need were observed.

Another less obvious use case is desorption of nano aerosol constituents to provide yet another dimension of the analysis to the system and the methodology.

FIG. 16 is illustrating calibration device assembly in/into the MPCI-system according to the present disclosure. Sample introduction port in FIG. 16 is providing a controlled way to introduce a gaseous sample into the IMR region. A valve to control calibrant gas injection into sample introduction port centerline. Calibration flow feed provides clean gas feed (Ultra Hight Purity Nitrogen or similar, clean gas) to the calibration vessel. Provided and controlled by an Mass Flow Controller or similar.

Calibration vessel assembly comprises body, calibrant vessel (vial, permeation tube or similar containing a calibrant compound such as Isopropanol), a heating element to heat up the calibration assembly to control calibrant flow saturation.

Alternatively a cooling element such as Peltier Thermo electric cooling element, advantageously keeping the calibration vessel temperature lower than ambient to avoid condensation in the saturated calibrant flow lines. A temperature measurement device (e.g. pt100) to provide temperature reading.

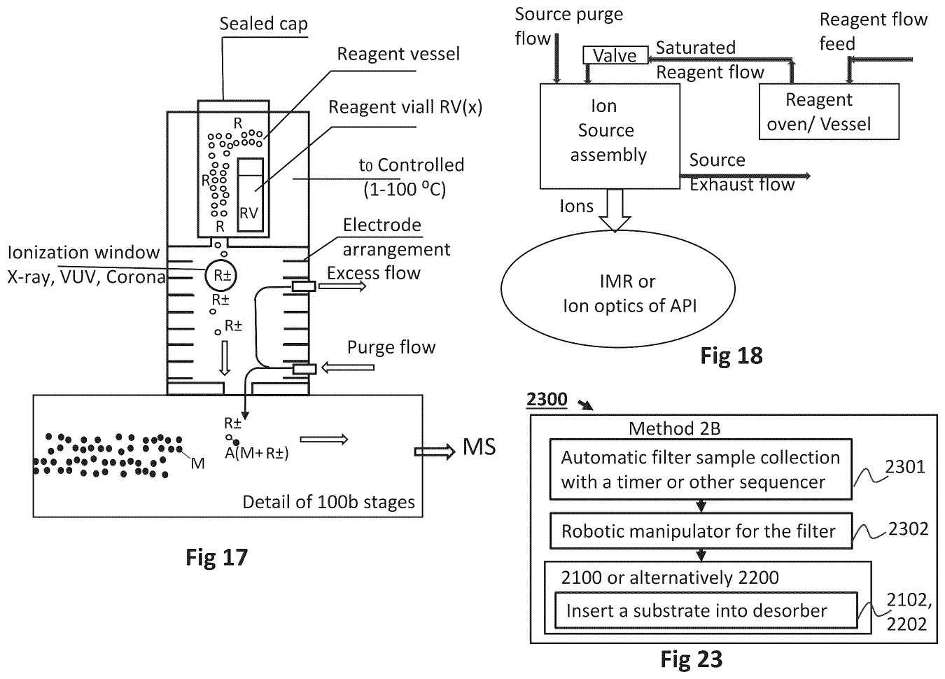

As illustrated in FIG. 17, such a set-up can be used in introducing samples, but also calibration substances in accordance of the embodiment detail of the system in FIG. 16.

In FIG. 17 embodiment, there is a temperature controlled reagent vessel where a reagent vial is placed. Temperature control is achieved by controlling heating elements and Peltier elements to heat and cool the volume under the control of a controller addressed to such a task, such as in FIGS. 4, 4A, 4B, 4C in addition to the other tasks of such.

This is done to control evaporation of the reagent from the vial, to speed up evaporation of low volatility compounds or slow down evaporation of high volatility compounds to produce a stable concentration of reagent vapor. Reagent vessel is sealed (in some embodiments, in alternative there could be a small feed through flow of clean gas) and connected with port (orifice) to the ionization volume which contains ionization window to ionize neutral reagent molecules, arrangement of electrodes to push ionized reagent into IMR (Ion-Molecular Reactor) against a purge flow and flow arrangement to create clean gas curtain to separate IMR from neutral reagent. With this kind of embodied arrangement, reagent is safely contained and propagated to the ionization window by means of diffusion according to principles of ideal gas law.

Reagent vial in the example is a container made of steel, glass or ceramics (chemically resistant and neutral material) and filled with reagent source FIGS. 17A, 17B and 17C examples: as in FIG. 17A) liquid, FIG. 17B) solid, FIG. 17C saturated porous material (e.g. molecular sieves, ceramic granules, steel granules) or combination of embodiments in FIG. 17A and FIG. 17C or FIG. 17B and FIG. 17C, the introduction of porous materials may increase reagent feed stability through capillary effects,

According to an embodiment variant, such Reagent source variants can be used as: 1) Nitric acid (or Citric Acid, Acetic Acid, Formic Acid)—to create NO3-, NO3-HNO3, NO3-(HNO3), 2 ions for adduct forming ionization of acidic and highly oxygenated compounds, 2) Dibromomethane to create Br- and Br[81](and/or Br[79])-ions for adduct forming and charge transfer ionization of less acidic and less oxygenated species, as well as HOO radicals, halogenated acids and alcohols, 3) Diethilamine (and or other amines) to create DEA-H+ (protonated reagent) for adduct forming and charge transfer ionization of less oxygenated species, volatile organic compounds, and compounds with high proton affinity, 4) Diketones (e.g. Acetonylacetone, Acetylacetone) to create AcAc-H+ (protonated reagent ion) for adduct forming ionization of basic compounds ammonia, amines, 5) Amides (e.g. Butyramide, Acetamide) to create BA-H+ (protonated reagent) for charge transfer and adduct forming ionization of a wide range of compounds, 6) Polyclic Aromatic Hydrocarbons (e.g. Fluoranthene) to create either protonated FLA-H+ reagent ion or a radical ion FLA+ (and or FLA−) for charge transfer ionization at reduced pressure for efficient and stable ionization of small VOCs, aromatic compounds and compounds with low proton affinity and simple ketones. Combination of these and other reagents in both high and low pressure ionization would give unprecedented breadth of analysis not achieved so far by any instrument. However, accordingly the example in FIG. 17 is illustrating chemical ionization of analytes (M) by a reagent R, being produced by the embodied ion source assembly in the FIG. 17, According to such a detail of the system as its element, substances such as Reagents can be introduced to the MPCI MION system. For example, the substance to be introduced in FIG. 17 is a reagent, symbolized by R, to denote to reagent as such. Although there is not shown a number (n), (m) or other character letter in the expression R, such as Rn or Rm, a skilled person in the art knows that any reagents used in the embodiments can be introduced similar way when applicable, The RV is representing a reagent comprising vial, and the marking RV(x) is illustrative of such a regent vial RV that has reagent x in a special form for the introduction to the system as released. Suitable form of the reagent as such are illustrated in FIGS. 17A, 17B, and 17C, from which the type for a particular reagent substance can be selected according to the purpose of the reagent to react with for adduct formation in chemical ionization process in the IMR- or API-stages (FIGS. 8 to 14), in the control of the control unit in question (such as shown in FIG. 4 and/or FIG. 4C).

The reagent vial in FIG. 17 example is in Reagent vessel, that has a controlled environment in respect to the pressure and temperature t0, in the example shown as been controlled between 1 and 100 C, for example. The values has been shown as an example, without intention to limit the temperature to the exemplified values only. Heating and cooling can be achieved by respective electric heaters and Peltier elements, for example, but other temperature control means can be used alternatively in suitable part, in the control of the controller shown in FIG. 4 and/or FIG. 4C.

The Reagent vessel can be sealed by Sealed cap, so that the reagent vessel can be serviced and maintained.

The small circles at the Reagent R are illustrating reagent molecules released from the Reagent vial, from the heat bath with the temperature t0 (1-100° C.). The Reagent vessel directs the reagent molecules R to a path leading to the ionization window, at the entry location to the electrode arrangement comprising ring-electrodes illustrated by the short horizontal lines at the sides of the cross section. The radiation window can be reagent specifically selected to pass through radiation, X-ray, beta, gamma, UV and/or can have an alfa emitter source stripe, for such embodiments where the reagent accelerator rings are held in underpressure conditions. The plus-minus symbols that the polarity is selectable according to the desired reagent and its use in the chemical ionization of the analyte molecules (marked as M, and the nearby black dots as such) in the IMR and/or API stages, before they are directed to the mass analysis by the Mass spectrometer MS in form of adducts A(M+R±), the marking symbolizing the formation by chemical ionization of the analyte M by the reagent R, having positive (+) or negative (−) polarity. The chemical ionization is selective to the analyte substances and utilized in the region of the IMR and/or API-stages at the reagent introduction region to enter to the IMR and/or API stage.

According to an embodiment variant, the charging can be obtained at the ionization window region by suitable electrodes and/or other charging means to be used for direct photoionization, corona discharge and Townsend ionization. Electrospray may be applicable in suitable part, under the control of the controller embodied in FIG. 4, 4A, 4B or 4C.

FIG. 17A is illustrative of a liquid form (l) reagent in the reagent vial RV(l). FIG. 17B is illustrative of a solid form (s) reagent in the reagent vial RV(s). The reagent can be the beads illustrated as such by the circles in the shown reagent vial cross section and/or on those beads that can be solid beads, but can have on the surfaces and/or in the intermediate spaces the reagent. The reagent can be evaporative and/or sublimate-released type. FIG. 17C is illustrative of a reagent vial RV(s,l,g), that has porous material beads, illustrated by the dashed line circles, to carry in the intermediate spaces the reagent, or on the porous surface bind reagent. A skilled person knows from these embodiment details of FIGS. 17A, 17B and 17C that it could be possible to use also mixed reagents from one vial as such, provided that the charging phenomena at the charging window (FIG. 17) as well as the chemical ionization as such are compatible to support the mass analysis in the Mass spectrometer. Source, purge and exhaust (Excess flow) flows are used as to create a clean gas curtain to prevent neutral reagent molecule leaving the source assembly. This is done to avoid doping of the sample with reagent gas, which would alter the composition of the sample gas and prevent efficient use of the other parallel ionization modes. It is also possible to flush and clean the ion source assembly in FIG. 17, to avoid contamination when the system needs to be shut off. In FIG. 18 (Slide 13) is illustrating in a schematic way a similar ion source assembly solution as shown in FIG. 17. The example is related to ion sources of the MPCI MION system to provide the charges and their carriers to the device of the present disclosure. Reagent oven/heater/furnace is to provide a space for reagent vessel (permeation tube or vial) and heating or cooling element arrangement to provide uniform temperature profile to the source arrangement and to control reagent evaporation rate (control by the controller similar to as shown in FIGS. 4 and/or 4C).

Source purge and exhaust flows are to create a clean gas curtain to prevent neutral reagent molecule leaving the source assembly.

Reagent flow feed provides clean carrier gas to Reagent oven/heater/furnace from the reagent vessel. From there evaporated reagent is carried to the ion source (i.e. ionization window region in FIG. 17) to be ionized and pushed to IMR or API stages, in the controlled conditions accordingly (FIG. 4, FIG. 4C).

Ion source could be dielectric barrier discharge, corona discharge, afterglow discharge, VUV (Vacuum UV-source), x-ray, Electrospray.

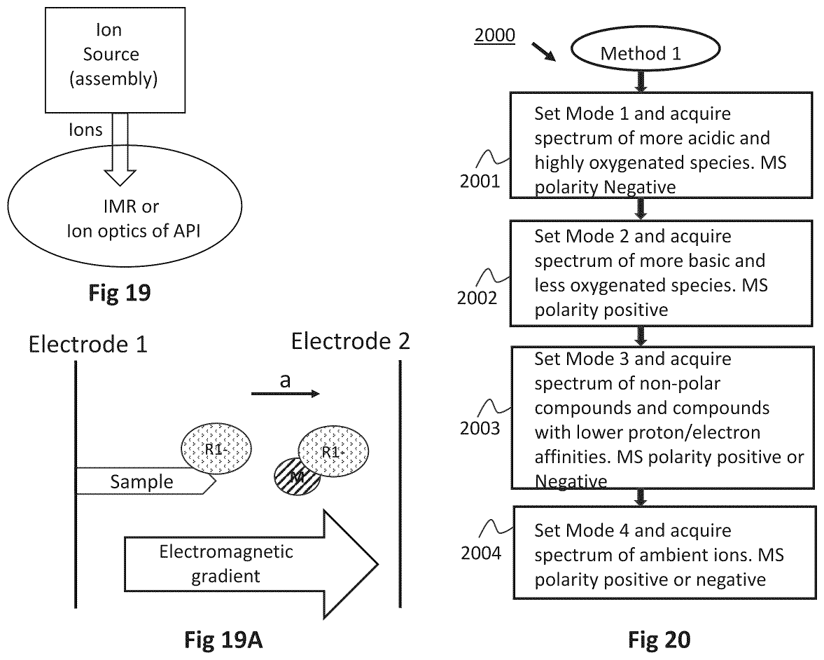

As illustrated in FIG. 19, according to such an alternative embodiment variant of the MPCI MION system detail of ion source injection, the MPCI MION system can use such a ion injection in which the ons are pushed towards sample centerline by means of an electric field created by an ion source or a special arrangement of electrodes

Accordingly, as illustrated by the sideview FIG. 19A, alternatively in the sample path there is an arrangement of electrodes (Electrode 1 and Electrode 2) creating an electromagnetic gradient to accelerate (a) created ions resulted in collision of reagent ions (R1-) and sample molecules (M) in the centerline region to advantageously vary collision energy and speed of extraction of the ions with varying voltages in electrode arrangement. Such an electrode arrangement can be controlled with a similar controller as in FIGS. 4 and/or 4C, as provided with means to control the electrode voltages illustrated.

In FIGS. 19 and 19A, the ion source as such can be embodied as illustrated in FIGS. 17 to 17C and/or FIG. 18.

FIG. 20 is illustrative a method embodiment example as Method 1 for using the disclosed Modes for the indicated purposes in the FIGS. 8 to 14, but also in FIGS. 15 to 19A in applicable part. In this illustrative example sample is in the gaseous form, entering an IMR. For example, the conditions corresponding or are that of ambient air. An acquisition of a full spectrum of compounds. For example for atmospheric research and Cloud Condensation Nuclei formation research, or breath analysis.

The performance of the MPCI MION system according to the Method 1 as illustrated can add a computer controlled method of switching and orchestrating ionization means and systems as embodied and illustrated according to the disclosure for the modes (FIGS. 8 to 14), as well as in FIGS. 15 to 19A in applicable part, modes with mass spectrometer polarity and tunings.

The Method 1 comprises phases as follows:

In phase 2001 the Method 1 comprises: Setting the MPCI MION system into Mode 1 and acquiring spectrum of more acidic (i.e. in acidic range of pH) and highly oxygenated molecules, i.e. oxidized molecules with large number of oxygen atoms (more than 4), while the MS has been controlled to negative polarity.

In phase 2002, Setting the MPCI MION system into Mode 2 and acquiring spectrum of more basic (i.e. in basic range of pH) and less oxygenated species (less than 4 oxygen atoms), while the MS
has been controlled to positive polarity.

In phase 2003, setting the MPCI MION system into Mode 3 and acquiring spectrum of non-polar, non-functionalized compounds and compounds with lower proton/electron affinities, such as VOC(Volatile Organic Compounds). The MS polarity being set positive or negative, or, if applicable to alter between the positive and negative polarities.

In phase 2004, setting the MPCI MION system into Mode 4 and acquiring spectrum of ambient ions. MS polarity being set positive or negative, or, if applicable to alter between the positive and negative polarities.

Accordingly the mass spectra acquired by the mass spectrometer MS are recorded and substances from the mass spectra identified and the composition of the sample being deduced. The mass-analysis results are recorded to the database for the gas sample and its constituents.

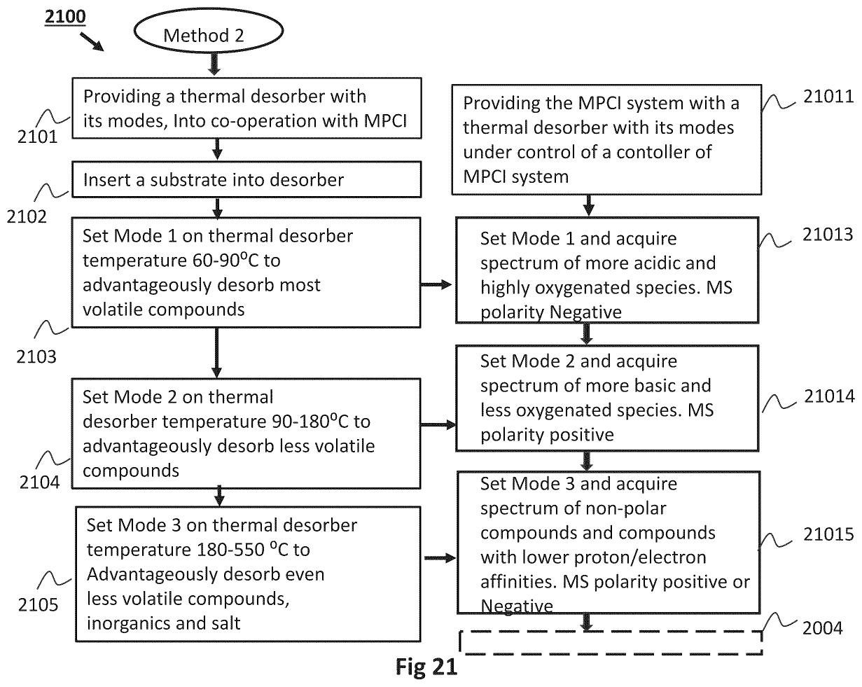

FIG. 21 is illustrative of an alternative variant of the Method 1 as being performed with such an alternative MPCI MION system that has a thermal desorber available in use in the MPCI MION system variant, able to switch between the modes of the thermal desorber in synchronism to the modes of MPCI MION device set-up according to the disclosure of the embodiment variants for the modes in FIGS. 8 to 14, under the control of a control unit embodied in FIG. 4 and/or FIG. 4C Such an alternatively arranged or tuned MCPI system can be used in for example by using the thermal desorber for analysis of filter media which should be considered, for instance for PM2.5, PM10 to make the chemical composition analysis of particulate matter or detection of explosives and/or illicit drugs by means of multiple ions and machine learning, as being utilized to detect and remember the mass-spectra acquired elemental compositions of the substances in the analyte molecules, in a mass-spectra database comprising the mass-analysis results as well as the identified compositions and/or the elemental isotopic variations in the identified compositions, so to provide information for tracing the origin in suitable extent.

Accordingly, acquisition of a full mass spectrum of compounds can be obtained. This is useful for example for environmental monitoring, security screening or contraband detection. In this embodiment thermal desorption modes are matched with MPCI MION device mode of mass spectrometer.

According to the present disclosure of the embodiments of the invention the alternative method to Method 1 as embodied as Method 2 (2100) comprises providing 2101 a thermal desorber with its modes of operation, into co-operation with the embodied MPCI-system. This is seen in the side of the MPCI MION system as providing 21011 the thermal desorber with its operational modes under the control of a controller of the MPCI MION system.

The mass analysis according to the Method 2 (2100) of the sample begins with the insertion of the sample substrate 2102 into the desorber. The desorber is set 2103 to its operational mode 1, Mode 1, to set Mode 1 on thermal desorber temperature 60-90° C. to advantageously desorb most volatile compounds. At the MCPI system the system itself with the mass spectrum acquisition line, (as in FIGS. 8 to 14), is set 21013 to its Mode 1 and the MPCI MION device in the system is acquiring a mass spectrum of more acidic (i.e. acidic pH and/or chemical behavior according to the acidity) and highly oxygenated species. Mass spectrometer MS polarity is set negative. The desorber is then set 2104 to its operational mode 2, Mode 2, to set in Mode 2 thermal desorber to temperature 90-180° C. to advantageously desorb less volatile (or intermediately volatile) compounds. At the MCPI system the system itself with the mass spectrum acquisition line, (as in FIGS. 8 to 14), is set 21014 to its Mode 2 and the MPCI MION device is acquiring a mass spectrum of more basic (i.e. basic pH and/or chemical behavior according to the base-nature) and less oxygenated species. Mass spectrometer MS polarity is set positive. The sample analysis continues in the example by setting 2105 the desorber to Mode 3 as its operational mode 3, Mode 3, to set in Mode 3 thermal desorber to temperature 180-550° C. to advantageously desorb even less volatile (or low volatile) compounds. At the MCPI system the system itself with the mass spectrum acquisition line, (as in FIGS. 8 to 14), is set 21015 to its Mode 3 and the MPCI MION device of the system is acquiring a mass spectrum of non-polar compounds and compounds with lower proton/electron affinities. MS polarity positive or Negative, or can be altered if applicable. At the end of the acquiring, the ambient ions can be acquired 2004. This can be made also before the setting of Mode 1 21013, or both ways, so to reveal potential contamination risks or need for maintenance/cleaning.

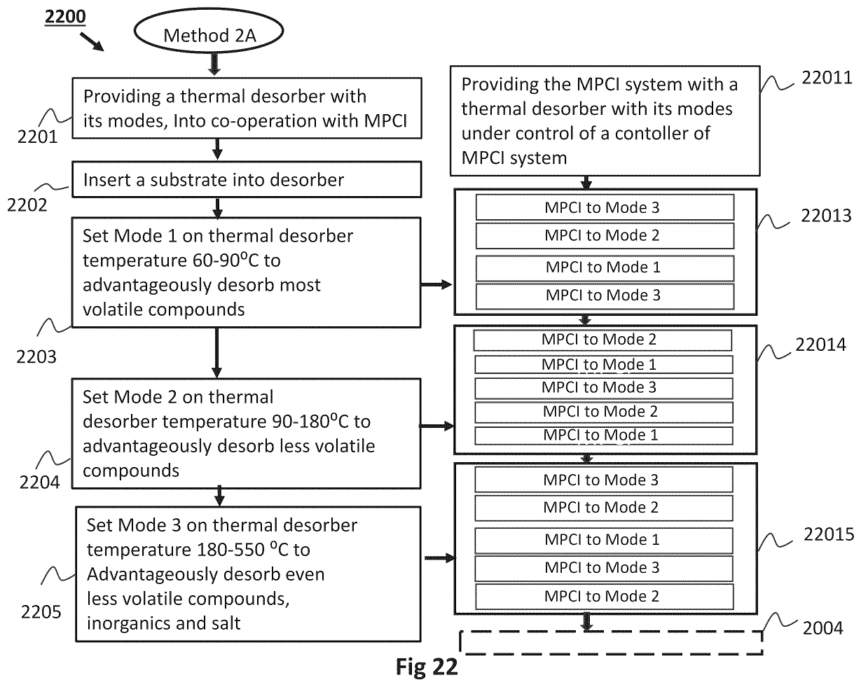

FIG. 22 is illustrating an alternative method variant Method 2A, to the Method 1, and/or to Method 2 embodiment illustrated in FIG. 21. Therein, in the FIG. 22 alternative embodiment, the thermal desorber operation is similar to the embodiment in FIG. 21, under the control of the MPCI MION device of the system controller (FIG. 4 and/or FIG. 4C). Accordingly in FIG. 22, the phase 2201 corresponds the phase 2101 in FIG. 21, the phase 2202 corresponds the phase 2102 in FIG. 21, the phase 2203 corresponds the phase 2103 in FIG. 21, the phase 2204 corresponds the phase 2104 in FIG. 21 and the phase 2205 corresponds the phase 2105 in FIG. 21. Also the phase 22011 in FIG. 22 corresponds the phase 21011 in FIG. 21.

In FIG. 22 embodiment, the MPCI MION mass spectrometer acquisition line is changing more rapidly than in the FIG. 21 example, to switch between Modes embodied in examples in FIGS. 8 to 14. The sample analysis according to the Method 2A 2200 continues via the phases 2203, 2204 and 2205 following the notation from the thermal desorber point of view, to utilize the MPCI MION DEVICE OF THE SYSTEM and the mass spectrometer therein in the scan through the phases 22011 to 22015, and optionally or in addition to have also similar or the phase 2004 at the end for the ambient ions to determine the baseline. According to an embodiment variant such a baseline type scan can be made in an operator selected phase, provided that the purge would not disturb the actual measurement going on in a whole.

In phase 2203 the thermal desorber Mode 1 as set to temperature 60-90° C. to advantageously desorb most volatile compounds, the MPCI MION devise of the system is set in the associated phase 22013 of the example to scan with the mass spectrometer a sequence of Mode 3, Mode 2, Mode 1 and Mode 3. The sequence in 22013 is shown as an example, which is not intended to limit the order and/or the number of the set modes to the shown order and/or number only.

In phase 2204 the thermal desorber Mode 2 as set to temperature 90-180° C. to advantageously desorb less volatile compounds, the MPCI device of the system is set in the associated phase 22014 of the example to scan with the mass spectrometer a sequence of Mode 2, Mode 1, Mode 3, Mode 2, and Mode 1. The sequence occurring in 22014 is shown as an example, which is not intended to limit the order and/or the number of the set Modes to the shown order and/or number only.

In phase 2205 the thermal desorber Mode 3 as set to temperature 90-180° C. to advantageously desorb less volatile compounds, the MPCI MION DEVICE OF THE SYSTEM is set in the associated phase 22015 of the example to scan with the mass spectrometer a sequence of Mode 3, Mode 2, Mode 1, Mode 3, and Mode 2. The sequence occurring in 22015 is shown as an example, which is not intended to limit the order and/or the number of the set Modes to the shown order and/or number only.

FIG. 23 is illustrative a further alternative variant of the Method 1, Method 2 and/or Method 2A, being used in a manner denoted as Method 2B (2300). In Method 2B, an automatic filter sampler is making a collection 2301 into a filter sample, as set by a timer or another sequencer for intermittent or alarm-based collection, so that there is also a robotic sample manipulator to prepare and move 2302 the filter from the collection device to the desorber. The Method 2B can then continue according to the Method 2 (2100) or optionally according to Method 2A (2200). The phases 2102 and 2202 are both denoting to inserting the substrate into the desorber, however in Method 2B this can be achieved by the same manipulator as in phase 2302, manually or by a dedicated loader to load such filters after the phase 2302 to deliver the filter to the thermal desorber.

Such an alternative Method 2B with a thermal desorber for analysis of filter media should be considered, for instance for PM2.5, PM10 chemical composition analysis or detection of explosives & illicit drugs by means of Set Mode 1 on thermal multiple ions and machine learning.

Furthermore, in Method 2B the filters can be collected continuously and automatically for continuous monitoring. Data is collected and stored on the server providing in one system a comprehensive view on chemical composition of air being filtrated by the collector device.

It is also possible to adsorb the gases to a gas collector in addition to the particle filter, so that alternatively or in addition, different substrates can be used for gas and particle phase analytes to provide even broader understanding of the fractions and their composition in the air. The gas-filters can be also handled similarly as the particle filters by a robotic manipulator, for inserting them into the thermal desorber, to be analysed according to at least one of the Method 2, Method 2A and Method 2B.

According to an embodiment variant, an MPCI-method by the MPCI MION system in multi-mode ion detection, in combination to one or more embodiments is comprising (ip referring to an ion polarity, positive + or negative −):
Sampling a sample into an ion molecular reactor (IMR), the sample comprising analyte molecules (M), from which an ensemble of analytes (M) as present are addressed to detection in mass spectrometry (MS),
Activating an ion source of reagents (R1), (R2), (R3) for ions (R1(ip)), (R2(ip)), (R3(ip)), with ion polarity (ip) to produce ions for ionization of substances in the sample and/or ionization of the reagents (R1), (R2), (R3) of the respective ion source,
ionizing at least part of said analytes (M) by exposing to direct ionization and/or via collision with reagent ions, forming adducts (MR1(ip)), (MR2(ip)) (MR3(ip)) from analytes (M) present in the sample and reagents (R1), (R2), (R3) in the corresponding adduct forming Mode X,
Selecting of the ionization adduct forming Mode X, in conduct of a control unit controlling the method procedure,
Analyzing the adducts MR1(ip)), (MR2(ip)) (MR3(ip)) and/or analyte ions (M(ip)) from the sample by a mass spectrometer (MS) according to the adduct forming Mode X.
According to an embodiment the MPCI-multi-mode ion detection method according to an embodiment utilizes the MPCI MION system modes for adduct forming in the mass spectrometry of a sample, wherein said adduct forming Mode X is one of the modes that comprises Mode 1, Mode 2, Mode 3, Mode 3B, Mode 3C, Mode 4, Mode 5,
wherein in the Mode 1 ions are produced by at least one ion tower in a stage of atmospheric pressure, while the rest of the ion towers are off,
wherein in the Mode 2 ions are produced by at least one ion tower different than in Mode 1, in same stage of atmospheric pressure as in Mode 1, while the rest of the ion towers are off,
wherein in the Mode 3 ions are produced by at least one ion tower in a stage of next low-pressure unit (B′) in the stack in reduced pressure conditions, while the rest of the ion towers are off, the ions produced are different ions than in Mode 1 or Mode 2,
wherein in the Mode 3B ions are not produced actively by the ion towers in the stages in the stack in atmospheric or reduced pressure conditions, as the ion towers are off, while measuring ambient ions.
wherein in the Mode 3C there are Mode 1 and Mode 3 towers controlled to on, at the same time. In such an embodiment of Mode 3C, for example positive mode in high pressure and positive mode ion source in low pressure, accordingly this is to ionize in both pressures simultaneously. However, it is possible to do the same in negative polarity where applicable.
wherein in the Mode 4 ions are not produced actively by the ion towers in the stages in the stack in atmospheric or reduced pressure conditions, as the ion towers are off. The mass spectrometer can perform zeroing of the MPCI-system and the stages, while measuring ambient ions from a selected source for the zeroing, so to check the system for the noise. This mode can be also considered as a calibration mode in accordance to the FIG. 14.
wherein in the Mode 5 ions are produced actively by the ion towers in two or more each-other-following underpressure stages (compartments) the stages are in reduced pressure conditions, in reducing pressures towards the mass spectrometer.

1. A Multi pressure chemical ionization multi-ion identification device, wherein said device comprises in a stack of ionization stages at least one atmospheric and/or high pressure ionization stage followed by at least one underpressure ionization stage in series following said at least one atmospheric and/or high pressure ionization stage in the device.

2. The Multi pressure chemical ionization multi-ion identification device according to claim 1, wherein, the MPCI-device further comprises a sample introduction port (100a) for sample with analyte molecules therein, said sample introduction port (100a) being followed by a stack (100b) of ionization stages (A, B, B′, C′, D′, E′), wherein said stack (100b) comprises at least one ionization stage in atmospheric or high pressure (A), (B) followed by a number of underpressure ionization stages (B′), (C′), (D′), (E′) in series of reducing operation pressure towards an exit to a mass spectrometer (MS) for a mass spectrum acquiring.

3. The multi pressure chemical ionization multi-ion identification device according to claim 1, wherein each ionization stages (A), (B), (B′), (C′), (D′), (E′) comprising a ionization-stage-specific number of ion injection towers (R1, R2,R3), (R4, R5,R6), (R7, R8, R9), (R10, R11,R12), (R13, R14,R15), (R16, R17, R18), each said ion injection tower being selected for a dedicated reagent (Rn), (Rm) emission in the ionized form at the entry to the corresponding ionization stage, for chemical ionization of analytes from the sample, in such a ionization stage in said ionization stage specific pressure lower than the high pressure stage pressure, and temperature condition.

4. The Multi pressure chemical ionization MPCI MION device according to claim 1 wherein in each ionization stage with its plurality of reagent ion towers (R1, R2, R3) to provide dedicated ionization reagent ions each, said each ionization stages are positioned into a stack as stacked to planar geometry with planes (IR(A), IR(B), IR(B′), IR(C′), IR(D′), IR(E′),) each ionization stages as mutually parallel while perpendicular to sample flow entrance direction to ionization region of the Multi pressure chemical ionization multi-ion identification device (100),

5. The Multi pressure chemical ionization device (MPCI-MION device) according to claim 1, wherein each ionization stage has a predetermined thickness in the sample flow direction correspond the timescale of chemical ionization in the pressure and temperature conditions of the ionization stage to provide a stage specific reaction time for the ions emitted from the respective ion towers to chemically ionize analyte molecules in the sample, the so formed adducts being transported from the ionization region to next stage or to a mass spectrometer port for mass analysis of the species of the adducts.

6. The MPCI-MION device according to claim 1, wherein each ionization reagent of the respective reagent ion injection tower in same plane (IR(A)) is configured to operate according to same ion production mechanism to provide respective reagent ions from a dedicated reagent ion tower.

7. The MPCI MION multi-ion identification device according to claim 1, wherein the ionization mechanism of a reagent ion tower to ionize reagent molecules to reagent ion in the ion towers by the ionization mechanism of the ion tower is at least one of the following: X-ray, soft-X-ray, corona discharge, electrospray, xenon UV lamp, based ionization mechanism.

8. The multi-ion identification device according to claim 1, wherein the polarity of a reagent ion tower produced reagent ions are adjustable to positive or negative ions.

9. The multi-ion identification device according to claim 1, wherein at least one of the reagent ion towers comprises a filter to filter away multiply charged reagent agents away from entry to the ionization region.

10. The multi-ion identification device according to claim 1, wherein the ionization region comprises a round cylindrical symmetry with a centerline (C) as a symmetry center of the stack (100b).

11. The multi-ion identification device according to claim 1, wherein the reagent ion towers in the same stage) are aligned into a corresponding plane (IR(A), IR(B), IR(B′), IR(C′), IR(D′), IR(E′),) and have an off-set (α) to deflect from the direction of the center line (C) along the respective emitting lines).

12. A MPCI MION multi-ion identification system (Sys) comprising following items:
at least one MPCI MION multi-ion identification device according to claim 1,
at least one control unit to control the MPCI MION multi-ion identification system and its actuators for the operation in mass analysis of adducts formed from the constituents of the sample,
a mass spectrometer (MS) to make said mass analysis,
a database (DB) to store and process mass analysis results.

13. The multi-ion identification system of claim 12, wherein the system comprises a software packet (SW) configured to control operation of the MPCI MION multi-ion identification system.

14. The MPCI MION multi-ion identification system of claim 12, wherein the system comprises such a software packet (SW) that is configured to make group analysis to find and deduce marker substances from the results.

15. The MPCI MION multi-ion identification system of claim 14, wherein the software packet (SW) comprises at least one of the following: a machine learning algorithm, a neuron network solver for classification and optimization of data clusters, an artificial intelligence algorithm, such as penalized linear LARS, an elastic net regressions algorithm, random forests and recursive feature elimination algorithm, to be used to analyze, compare and predict chemical features of gaseous samples.

16. The MPCI MION system of claim 12, wherein the system comprises an ion detector to detect ions, said ion detector being configured to simultaneously utilize multiple selective ion chemistries both in negative and positive modes of detection.

17. The MPCI MION system of claim 12, wherein the system is configured to detect extremely low vapor pressure, highly oxidized multifunctional organic molecules (HOM) from the sample.

18. A method identifying substances from a gas sample by using a multi-ion system (MPCI-system) of claim 12, comprising:
sampling a gas sample into a sample flow of the MPCI MION multi-ion 10identification device,
allowing turbulence to decay to laminar flow conditions of the sample flow in a buffering region of the multi-ion identification device in the first stage,
protecting the gas sample by at least one or two sheath flows at least in the 15buffering region,
charging the gas sample constituents by reagent ion molecules formed for use in chemical ionization of said gas sample constituents to form adducts in corresponding stages with stage specific pressure and temperature,
allowing the adduct to form from the gas sample constituents and reagent 20ion molecules in corresponding stages,
leading the adducts to mass spectrometer for mass analysis,
identifying the adducts and the gas sample constituents,
storing to a database the identified gas sample constituents.

19. A mass spectrometer integrated together from the operable according to method claim 18.
